(19) United States
(12) Patent Application Publication
(10) Pub. No.: US 2024/0035003 A1
(43) Pub. Date: Feb. 1, 2024

(54) METHODS OF PRODUCING ADENOVIRUS

(71) Applicant: ASTRAZENECA UK LIMITED, CAMBRIDGE (GB)

(72) Inventors: JINLIN JIANG, GAITHERSBURG, MD (US); NICOLE BLECKWENN, GAITHERSBURG, MD (US); RAGHAVAN VENKAT, GAITHERSBURG, MD (US); DANIEL PAPPAS, GAITHERSBURG, MD (US); BENJAMIN RUSH, GAITHERSBURG, MD (US); GEORGE CHACKO, GAITHERSBURG, MD (US)

(21) Appl. No.: 18/256,484

(22) PCT Filed: Dec. 10, 2021
(86) PCT No.: PCT/EP2021/085192

§ 371 Date: Jun. 8, 2023

Related U.S. Application Data
(60) Provisional application No. 63/123,570, filed on Dec. 10, 2020.

(51) Int. Cl. C12N 7/02; C12N 15/86; A61K 39/215

(52) U.S. Cl. CPC C12N 7/02; C12N 15/86; A61K 39/215; A61K 2039/5254

(57) ABSTRACT

Methods for the production of adenoviruses which are suitable for use in a vaccine, and methods for increasing the yield of adenoviruses during production. These methods include adding an adenovirus to a cell population in culture; culturing the cell population under conditions which are permissive for infection of the cell population with the adenovirus to provide a cell population comprising adenovirus-infected cells; culturing the cell population comprising adenovirus-infected cells under conditions which are permissive for replication of the adenovirus; and harvesting the adenovirus from the culture.

### FIELD OF THE INVENTION

The present invention relates to methods for the production of adenoviruses. More particularly, the invention relates to methods for the production of adenoviruses which are suitable for use in a vaccine, and to methods for increasing the yield of adenoviruses during production.

### BACKGROUND OF THE INVENTION

Adenoviruses are double-stranded DNA viruses with a genome of approximately 26-46 kb. Adenoviruses are species-specific and different serotypes have been isolated from a variety of mammalian species. Human adenoviruses are ubiquitous, and most people have been infected with one or more serotypes, leading to lifelong immunity.

Modified adenoviruses can be used as vectors to deliver DNA coding for foreign antigens. Such adenovirus vectors are often replication-defective adenovirus vectors which have the essential E1A and E1B genes deleted and replaced by an expression cassette with a high activity promoter such as the cytomegalovirus immediate early promoter which drives expression of a heterologous gene.

Replication deficient adenovirus vectors have been employed extensively for vaccines because they induce a strong humoral and T cell response to the heterologous gene encoded by the vector. Results of a clinical trial investigating a replication deficient Ad5-based vaccine for use in the treatment of tuberculosis appears very promising (Smail et al. 2013; Sci. Transl. Med. October 2; 5(205):205ra134). Despite this, current methods for production of such adenoviruses are inefficient and lack scalability.

Therefore, there exists a need for improved methods for the production of adenoviruses.

### SUMMARY OF THE INVENTION

The present invention relates, at least in part, to the development of improved adenovirus production methods which are highly scalable and provide increased adenovirus vector titer compared with alternative production methods. The methods of the present invention are therefore highly advantageous, in particular where large quantities of adenovirus vectors are required, such as for the provision of adenovirus-based vaccines for epidemic and pandemic diseases.

Accordingly, in one aspect, there is provided a method of producing an adenovirus for use in a vaccine, the method comprising:
(a) adding an adenovirus to a cell population in culture at an MOI insufficient for infection of all the cells in the cell population;
(b) culturing the cell population under conditions which are permissive for infection of the cell population with the adenovirus to provide a cell population comprising adenovirus-infected cells;
(c) culturing the cell population comprising adenovirus-infected cells under conditions which are permissive for replication of the adenovirus; and
(d) harvesting the adenovirus from the culture.

In another aspect, there is provided a method of producing an adenovirus for use in a vaccine, the method comprising culturing a cell population comprising a first fraction of adenovirus-infected cells under conditions which are permissive for infection of a second fraction of the cell population with the adenovirus, wherein the second fraction of the cell population is infected by adenovirus released by the first fraction of adenovirus-infected cells.

In yet another aspect, there is provided a method of producing an adenovirus for use in a vaccine, the method comprising:
(a) adding an adenovirus to a cell population in culture;
(b) culturing the cell population under conditions which are permissive for infection of the cell population to provide a cell population comprising adenovirus-infected cells;
(c) culturing the cell population comprising adenovirus-infected cells under conditions which are permissive for replication of the adenovirus; and
(d) harvesting adenovirus from the culture about 96-144 hours after adding adenovirus to the cell population.

In another aspect, there is provided a method of producing an adenovirus for use in a vaccine, the method comprising:
(a) seeding cells in a cell culture vessel at an initial cell density of at least 0.5×10⁶ cells/mL to provide a cell population in culture;
(b) adding an adenovirus to the cell population in culture;
(c) culturing the cell population under conditions which are permissive for infection of the cell population to provide a cell population comprising adenovirus-infected cells;
(d) culturing the cell population comprising adenovirus-infected cells under conditions which are permissive for replication of the adenovirus; and
(e) harvesting adenovirus from the culture.

In another aspect, there is provided a method of producing an adenovirus for use in a vaccine, the method comprising:
(a) adding an adenovirus to a cell population in culture having a viable cell density of at least about 1×10⁶ cells/mL;
(b) culturing the cell population under conditions which are permissive for infection of the cell population with the adenovirus to provide a cell population comprising adenovirus-infected cells;
(c) culturing the cell population comprising adenovirus-infected cells under conditions which are permissive for replication of the adenovirus; and
(d) harvesting the adenovirus from the culture.

In another aspect, there is provided a method of producing an adenovirus for use in a vaccine, the method comprising:
(a) adding an adenovirus to a cell population in culture having a viable cell density of at least about 1×10⁶ cells/mL at an MOI insufficient for infection of all the cells in the cell population;
(b) culturing the cell population under conditions which are permissive for infection of the cell population to provide a cell population comprising adenovirus-infected cells;
(c) culturing the cell population comprising adenovirus-infected cells under conditions which are permissive for replication of the adenovirus; and
(d) harvesting adenovirus from the culture about 96-144 hours after adding adenovirus to the cell population, wherein the method comprises switching the temperature to which the cell population is exposed from a first temperature to a second temperature, wherein the first and second temperatures are permissive for infection of the cell population with the adenovirus.

In another aspect, there is provided a method of producing an adenovirus for use in a vaccine, the method comprising:
(a) adding an adenovirus to a cell population in culture having a viable cell density of at least about 1×10⁶ cells/mL at an MOI insufficient for infection of all the cells in the cell population;
(b) culturing the cell population under conditions which are permissive for infection of a first fraction of cells in the cell population with the adenovirus;
(c) culturing the cell population comprising the first fraction of adenovirus-infected cells under conditions which are permissive for infection of a second fraction of cells in the cell population with the adenovirus, wherein the second fraction of cells is infected by adenovirus released into the culture by the first fraction of adenovirus-infected cells;
(d) culturing the cell population comprising adenovirus-infected cells under conditions which are permissive for replication of the adenovirus; and
(e) harvesting the adenovirus from the culture about 96-144 hours after adding adenovirus to the cell population, wherein the method comprises switching the temperature to which the cell population is exposed from a first temperature to a second temperature, wherein the first and second temperatures are permissive for infection of the cell population with the adenovirus.

In another aspect, there is provided a method for preparing a vaccine comprising an adenovirus, the method comprising:
(a) adding an adenovirus to a cell population in culture at an MOI insufficient for infection of all the cells in the cell population;
(b) culturing the cell population under conditions which are permissive for infection of the cell population to provide a cell population comprising adenovirus-infected cells;
(c) culturing the cell population comprising adenovirus-infected cells under conditions which are permissive for replication of the adenovirus;
(d) harvesting the adenovirus from the culture;
(e) purifying the adenovirus; and
(f) preparing a vaccine comprising the purified adenovirus.

In another aspect, there is provided a method for preparing a vaccine comprising an adenovirus, the method comprising:
(a) culturing a cell population comprising a first fraction of adenovirus-infected cells under conditions which are permissive for infection of a second fraction of the cell population with the adenovirus, wherein the second fraction of the cell population is infected by adenovirus released into the culture by the first fraction of adenovirus-infected cells;
(b) harvesting the adenovirus from the culture;
(c) purifying the adenovirus; and
(d) preparing a vaccine comprising the purified adenovirus.

In another aspect, there is provided a method for preparing a vaccine comprising an adenovirus, the method comprising:
(a) adding an adenovirus to a cell population in culture;
(b) culturing the cell population under conditions which are permissive for infection of the cell population to provide a cell population comprising adenovirus-infected cells;
(c) culturing the cell population comprising adenovirus-infected cells under conditions which are permissive for replication of the adenovirus;
(d) harvesting adenovirus from the culture about 96-144 hours after adding adenovirus to the cell population;
(e) purifying the adenovirus; and
(f) preparing a vaccine comprising the purified adenovirus.

In another aspect, there is provided a method for preparing a vaccine comprising an adenovirus, the method comprising:
(a) seeding cells in a cell culture vessel at an initial cell density of at least 0.5×10⁶ cells/mL to provide a cell population in culture;
(b) adding an adenovirus to the cell population in culture;
(c) culturing the cell population under conditions which are permissive for infection of the cell population to provide a cell population comprising adenovirus-infected cells;
(d) culturing the cell population comprising adenovirus-infected cells under conditions which are permissive for replication of the adenovirus; and
(e) harvesting adenovirus from the culture;
(f) purifying the adenovirus; and
(g) preparing a vaccine comprising the purified adenovirus.

In another aspect, there is provided a method for preparing a vaccine comprising an adenovirus, the method comprising:
(a) adding an adenovirus to a cell population in culture having a viable cell density of at least about 1×10⁶ cells/mL;
(b) culturing the cell population under conditions which are permissive for infection of the cell population with the adenovirus to provide a cell population comprising adenovirus-infected cells;
(c) culturing the cell population comprising adenovirus-infected cells under conditions which are permissive for replication of the adenovirus;
(d) harvesting the adenovirus from the culture;
(e) purifying the adenovirus; and
(f) preparing a vaccine comprising the purified adenovirus.

In another aspect, there is provided a method for preparing a vaccine comprising an adenovirus, the method comprising:
(a) adding an adenovirus to a cell population in culture having a viable cell density of at least about 1×10⁶ cells/mL at an MOI insufficient for infection of all the cells in the cell population;
(b) culturing the cell population under conditions which are permissive for infection of the cell population to provide a cell population comprising adenovirus-infected cells;
(c) culturing the cell population comprising adenovirus-infected cells under conditions which are permissive for replication of the adenovirus;
(d) harvesting adenovirus from the culture about 96-144 hours after adding adenovirus to the cell population;
(e) purifying the adenovirus; and
(f) preparing a vaccine comprising the purified adenovirus,
wherein the method comprises switching the temperature to which the cell population is exposed from a first temperature to a second temperature, wherein the first and second temperatures are permissive for infection of the cell population with the adenovirus.

In another aspect, there is provided a method for preparing a vaccine comprising an adenovirus, the method comprising:
(a) adding an adenovirus to a cell population in culture having a viable cell density of at least about 1×10⁶ cells/mL at an MOI insufficient for infection of all the cells in the cell population;
(b) culturing the cell population under conditions which are permissive for infection of a first fraction of cells in the cell population with the adenovirus;
(c) culturing the cell population comprising the first fraction of adenovirus-infected cells under conditions which are permissive for infection of a second fraction of cells in the cell population with the adenovirus, wherein the second fraction of cells is infected by adenovirus released into the culture by the first fraction of adenovirus-infected cells;
(d) culturing the cell population comprising adenovirus-infected cells under conditions which are permissive for replication of the adenovirus;
(e) harvesting the adenovirus from the culture about 96-144 hours after adding adenovirus to the cell population,
(f) purifying the adenovirus; and
(g) preparing a vaccine comprising the purified adenovirus, wherein the method comprises switching the temperature to which the cell population is exposed from a first temperature to a second temperature, wherein the first and second temperatures are permissive for infection of the cell population with the adenovirus.

In another aspect, there is provided a method for increasing the yield of an adenovirus during production of the adenovirus, the method comprising culturing a cell population in culture in the presence of an adenovirus at a first temperature and switching the temperature to which the cell population is exposed to a second temperature, wherein the first and second temperatures are permissive for infection of the cell population with the adenovirus.

In another aspect, there is provided a method for producing an adenovirus as set forth in FIG. 2.

In another aspect, there is provided an adenovirus for use in a vaccine, obtainable by or obtained by a method of the invention.

In another aspect, there is provided a vaccine comprising an adenovirus, obtainable by or obtained by a method of the invention.

In any of the aspects described herein, the method may comprise switching the temperature to which the cell population is exposed from a first temperature to a second temperature, wherein the first and second temperatures are permissive for infection of the cell population with the adenovirus.

Aspects and embodiments of the invention are set out in the appended claims. These and other aspects and embodiments of the invention are also described herein.

### BRIEF DESCRIPTION OF THE FIGURES

FIG. 1 shows an exemplary high MOI process. According to this method, cells are grown until they reach a confluency of approximately 3-5×10⁶ cells/mL at which point they are diluted 1:1 and infected with adenovirus at an MOI of 10. The infected cells are cultured for a further 42±2 hours before being harvested for adenovirus purification.

FIG. 2 shows an exemplary low MOI process according to the invention. Cells are seeded and infected with adenovirus at a low MOI of up to 1 approximately 24 hours after seeding. The infected cells are cultured for approximately 6 days before being harvested for adenovirus purification.

FIG. 3 shows a comparison of viable cell density (VCD), viability, and adenovirus titer during high and low MOI processes. FIG. 3A VCD; FIG. 3B viability; FIG. 3C qPCR titer; FIG. 3D A260:A280 ratio.

FIG. 4 provides a comparison of a high MOI process and a low MOI process. FIG. 4A viral genome concentration; infectious titer; and viral particle titer. FIG. 4B viral genome:infectious units ratio and A260:A280 ratio.

FIG. 5 shows the effect of MOI on VCD, viability and adenovirus titer. FIG. 5A VCD; FIG. viability; FIG. 5C adenovirus qPCR titer.

an MOI FIG. 6 shows adenovirus titer at different initial cell seeding densities and infection time points. FIG. 6A day 0 infection; FIG. 6B day 1 infection.

FIG. 7 shows the effect of dilution on viral titer in the low MOI process with infection at different time points. FIG. 7A day 0 infection; FIG. 7B day 1 infection.

FIG. 8 shows the effect of various cell culture additives on infectious titer.

FIG. 9 shows the effect of temperature shift on VCD, viability and adenovirus titer. FIG. 9A VCD; FIG. 9B viability; FIG. 9C adenovirus qPCR titer.

FIG. 10 shows the scalability of an exemplary low MOI process. FIG. 10A VCD; FIG. 10B viability; FIG. 10C adenovirus qPCR titer.

### DESCRIPTION OF SEQUENCE LISTING

| SEQ | | |
|---|---|---|
| ID NO: | Description | Sequence |
| 1 | Amino acid sequence of the | MFVFLVLLPL VSSQCVNLTT RTQLPPAYTN |
| | spike protein of the SARS- | SFTRGVYYPD KVFRSSVLHS TQDLFLPFFS |
| | CoV-2 strain of the SARS- | NVTWFHAIHV SGTNGTKRFD NPVLPFNDGV |
| | CoV species of coronavirus | YFASTEKSNI IRGWIFGTTL DSKTQSLLIV |
| | | NNATNVVIKV CEFQFCNDPF LGVYYHKNNK |
| | | SWMESEFRVY SSANNCTFEY VSQPFLMDLE |
| | | GKQGNFKNLR EFVFKNIDGY FKIYSKHTPI |
| | | NLVRDLPQGF SALEPLVDLP IGINITRFQT |
| | | LLALHRSYLT PGDSSSGWTA GAAAYYVGYL |
| | | QPRTFLLKYN ENGTITDAVD CALDPLSETK |
| | | CTLKSFTVEK GIYQTSNFRV QPTESIVRFP |
| | | NITNLCPFGE VFNATRFASV YAWNRKRISN |
| | | CVADYSVLYN SASFSTFKCY GVSPTKLNDL |
| | | CFTNVYADSF VIRGDEVRQI APGQTGKIAD |
| | | YNYKLPDDFT GCVIAWNSNN LDSKVGGNYN |
| | | YLYRLFRKSN LKPFERDIST EIYQAGSTPC |
| | | NGVEGENCYF PLQSYGFQPT NGVGYQPYRV |
| | | VVLSFELLHA PATVCGPKKS TNLVKNKCVN |
| | | FNFNGLTGTG VLTESNKKFL PFQQFGRDIA |
| | | DTTDAVRDPQ TLEILDITPC SFGGVSVITP |
| | | GTNTSNQVAV LYQDVNCTEV PVAIHADQLT |
| | | PTWRVYSTGS NVFQTRAGCL IGAEHVNNSY |
| | | ECDIPIGAGI CASYQTQTNS PRRARSVASQ |
| | | SIIAYTMSLG AENSVAYSNN SIAIPTNFTI |
| | | SVTTEILPVS MTKTSVDCTM YICGDSTECS |
| | | NLLLQYGSFC TQLNRALTGI AVEQDKNTQE |
| | | VFAQVKQIYK TPPIKDFGGF NFSQILPDPS |
| | | KPSKRSFIED LLFNKVTLAD AGFIKQYGDC |
| | | LGDIAARDLI CAQKFNGLTV LPPLLTDEMI |
| | | AQYTSALLAG TITSGWTFGA GAALQIPFAM |
| | | QMAYRFNGIG VTQNVLYENQ KLIANQFNSA |
| | | IGKIQDSLSS TASALGKLQD VVNQNAQALN |
| | | TLVKQLSSNF GAISSVLNDI LSRLDKVEAE |
| | | VQIDRLITGR LQSLQTYVTQ QLIRAAEIRA |
| | | SANLAATKMS ECVLGQSKRV DFCGKGYHLM |
| | | SFPQSAPHGV VFLHVTYVPA QEKNFTTAPA |
| | | ICHDGKAHFP REGVFVSNGT HWFVTQRNFY |
| | | EPQIITTDNT FVSGNCDVVI GIVNNTVYDP |
| | | LQPELDSFKE ELDKYFKNHT SPDVDLGDIS |
| | | GINASVVNIQ KEIDRLNEVA KNLNESLIDL |
| | | QELGKYEQYI KWPWYIWLGF IAGLIAIVMV |
| | | TIMLCCMTSC CSCLKGCCSC GSCCKFDEDD |
| | | SEPVLKGVKL HYT |
| 2 | Amino acid sequence of the | MDAMKRGLCC VLLLCGAVFV SASQEIHARF |
| | spike protein of the SARS- | RRFVFLVLLP LVSSQCVNLT TRTQLPPAYT |
| | CoV-2 strain of the SARS- | NSFTRGVYYP DKVFRSSVLH STQDLFLPFF |
| | CoV species of coronavirus | SNVTWFHAIH VSGTNGTKRF DNPVLPFNDG |
| | with the signal peptide of the | VYFASTEKSN IIRGWIFGTT LDSKTQSLLI |
| | human tissue plasminogen | VNNATNVVIK VCEFQFCNDP FLGVYYHKNN |
| | activator gene (tPA) at the N- | KSWMESEFRV YSSANNCTFE YVSQPFLMDL |
| | terminus | EGKQGNFKNL REFVFKNIDG YFKIYSKHTP |
| | | INLVRDLPQG FSALEPLVDL PIGINITRFQ |
| | | TLLALHRSYL TPGDSSSGWT AGAAAYYVGY |
| | | LQPRTFLLKY NENGTITDAV DCALDPLSET |
| | | KCTLKSFTVE KGIYQTSNFR VQPTESIVRF |
| | | PNITNLCPFG EVFNATRFAS VYAWNRKRIS |
| | | NCVADYSVLY NSASFSTFKC YGVSPTKLND |
| | | LCFTNVYADS FVIRGDEVRQ IAPGQTGKIA |
| | | DYNYKLPDDF TGCVIAWNSN NLDSKVGGNY |
| | | NYLYRLFRKS NLKPFERDIS TEIYQAGSTP |
| | | CNGVEGFNCY FPLQSYGFQP TNGVGYQPYR |
| | | VVVLSFELLH APATVCGPKK STNLVKNKCV |
| | | NFNFNGLTGT GVLTESNKKF LPFQQFGRDI |
| | | ADTTDAVRDP QTLEILDITP CSFGGVSVIT |
| | | PGTNTSNQVA VLYQDVNCTE VPVAIHADQL |
| | | TPTWRVYSTG SNVFQTRAGC LIGAEHVNNS |
| | | YECDIPIGAG ICASYQTQTN SPRRARSVAS |
| | | QSIIAYTMSL GAENSVAYSN NSIAIPTNFT |
| | | ISVTTEILPV SMTKTSVDCT MYICGDSTEC |
| | | SNLLLQYGSF CTQLNRALTG IAVEQDKNTQ |
| | | EVFAQVKQIY KTPPIKDFGG FNFSQILPDP |
| | | SKPSKRSFIE DLLFNKVTLA DAGFIKQYGD |
| | | CLGDIAARDL ICAQKFNGLT VLPPLLTDEM |
| | | IAQYTSALLA GTITSGWTFG AGAALQIPFA |
| | | MQMAYRFNGI GVTQNVLYEN QKLIANQFNS |
| | | AIGKIQDSLS STASALGKLQ DVVNQNAQAL |
| | | NTLVKQLSSN FGAISSVIND ILSRLDKVEA |
| | | EVQIDRLITG RLQSLQTYVT QQLIRAAEIR |
| | | ASANLAATKM SECVLGQSKR VDFCGKGYHL |
| | | MSFPQSAPHG VVFLHVTYVP AQEKNFTTAP |
| | | AICHDGKAHF PREGVFVSNG THWFVTQRNF |
| | | YEPQIITTDN TFVSGNCDVV IGIVNNTVYD |
| | | PLQPELDSFK EELDKYFKNH TSPDVDLGDI |
| | | SGINASVVNI QKEIDRLNEV AKNLNESLID |
| | | LQELGKYEQY IKWPWYIWLG FIAGLIAIVM |
| | | VTIMLCCMTS CCSCLKGCCS CGSCCKFDED |
| | | DSEPVLKGVK LHYT |

### DETAILED DESCRIPTION OF THE INVENTION

Adenovirus Production

Adenoviruses are non-enveloped viruses with linear, double stranded DNA (dsDNA) genomes between 26-46 kb in length. Replication incompetent adenovirus vectors have been used as vaccine vectors to deliver infectious pathogen antigens in multiple clinical trials. However, current methods for production of adenoviruses for use in a vaccine lack scalability and are limited by their final adenovirus titer. The present inventors have surprisingly found new adenovirus production methods which are highly scalable and provide increased adenovirus vector titer compared with alternative methods, making them appropriate choices for the production of adenovirus vector for inclusion in adenovirus-based vaccines for epidemic and pandemic diseases.

It will be understood that the methods of the invention may be for the production of adenovirus for use in a vaccine, e.g. for use in a COVID-19 vaccine.

The present inventors have surprisingly shown that adenovirus added to a cell population at a low multiplicity of infection (MOI) may provide a high virus titer (see Examples 1-2). Furthermore, product derived from the low MOI process had a comparable quality to that derived from an exemplary high MOI process (see Example 1). As will be readily appreciated, use of a low MOI process considerably reduces the viral seed requirement compared with a high MOI process. As used herein “MOI” refers to the ratio of the number of infectious virus particles to number of target cells in a cell population.

Accordingly, in some embodiments, the methods of the invention comprise adding an adenovirus to a cell population in culture. In preferred embodiments, the methods of the invention comprise adding an adenovirus to a cell population in culture at an MOI insufficient for infection of all the cells in the cell population. In some embodiments, the MOI is from about 0.003 to about 1, preferably from about 0.03 to about 0.3, most preferably about 0.1. For example, in some embodiments, the MOI is 0.025, 0.030, 0.052, 0.090, 0.100, 0.120, 0.180 or 0.270.

In some embodiments, prior to adding an adenovirus to the cell population in culture, the methods of the invention comprise seeding cells in a cell culture vessel to provide a cell population in culture. As used herein, a “cell culture vessel” refers to a container suitable for culturing cells. In some embodiments, a cell culture medium is used for cell seeding. As used herein, “cell culture medium” means a liquid solution that contains cell culture nutrients and salts used in the initial cell seeding step which is designed to support the growth and viability of cells in culture. In some embodiments, the cell culture medium used for cell seeding is BalanCD® HEK medium. In some embodiments, the cell culture medium used for cell seeding is not 293 SFM II.

Adenovirus may be added to cells at various time points after seeding the cells in a cell culture vessel. Accordingly, in some embodiments, the method comprises adding an adenovirus to a cell population in culture about 0-48 hours after seeding the cells in the cell culture vessel, preferably about 24 hours after seeding the cells in the cell culture vessel. For example, the adenovirus may be added to a cell population in culture within 6 hours, within 12 hours, within 18 hours, within 24 hours, within 32 hours, or within 48 hours after seeding the cells in the cell culture vessel.

Prior art approaches to adenovirus production have relied on using cell densities in the range of 5×10⁵ cells/mL at the time of infection, as using cell densities higher than this abolishes infectious particle production (reviewed in Kamen & Henry 2004 J. Gene Med. 2004 February; 6 Suppl 1:S184-92). Here, the present inventors have shown that increasing seeding cell density surprisingly increases viral titer (see Example 3). In preferred embodiments, the method comprises seeding cells in a cell culture vessel at an initial cell density of at least about 0.5×10⁶ cells/mL, preferably at least about 0.8×10⁶ cells/mL, most preferably at least about 1.2×10⁶ cells/mL. Increasing the initial cell seeding density may provide for an increased viable cell density of the cell population at the time of infection. In some embodiments, the method comprises adding an adenovirus to a cell population in culture having a viable cell density of at least about 0.5×10⁶ cells/mL, preferably at least about 0.75×10⁶ cells/mL, at least about 1×10⁶ cells/mL, at least about 1.5×10⁶ cells/mL, at least about 2×10⁶ cells/mL, or at least 2.5×10⁶ cells/mL, most preferably at least about 1×10⁶ cells/mL. In some embodiments, the method comprises adding an adenovirus to a cell population in culture having a viable cell density of from about 0.5×10⁶ cells/mL to about 1×10⁷ cells/mL, preferably from about 0.5×10⁶ cells/mL to about 5×10⁶ cells/mL, most preferably from about 0.5×10⁶ cells/mL to about 2.5×10⁶ cells/mL.

After addition of adenovirus to the cell population, adenovirus particles will attach to target cells before being endocytosed thereby infecting the target cells. Thus, in some embodiments, the methods of the invention comprise culturing the cell population under conditions which are permissive for infection of the cell population with the adenovirus to provide a cell population comprising adenovirus-infected cells. As used herein, “conditions which are permissive for infection” means any suitable manner of culturing a cell that permits entry of adenoviral DNA into the cell. Such conditions will depend on the cell population being cultured and the adenovirus used to infect the cells. Techniques for determining entry of adenoviral DNA into a cell are well known in the art and include qPCR.

When using a low MOI to infect a cell population, there may not be enough virus particles to infect all of the cells in the cell population. Accordingly, in some embodiments, the methods of the invention comprise culturing the cell population under conditions which are permissive for infection of a first fraction of cells in the cell population with the adenovirus. In some embodiments, the method further comprises culturing the cell population comprising the first fraction of infected cells under conditions which are permissive for infection of a second fraction of cells in the cell population with the adenovirus, wherein the second fraction of cells is infected by adenovirus released into the culture by the first fraction of infected cells. Accordingly, in preferred embodiments, the method is characterised by a first infection and a second infection, wherein the first infection provides a first fraction of adenovirus-infected cells and is induced by adding the adenovirus to the cell population, and wherein the second infection provides a second fraction of adenovirus-infected cells and is induced by adenovirus released into the culture by the first fraction of adenovirus-infected cells. In some embodiments, the conditions which are permissive for infection of the first and second fraction of the cell population are the same. In some embodiments, the conditions which are permissive for infection of the first and second fraction of the cell population are different. In some embodiments, the method comprises culturing the cell population comprising the first and second fraction of infected cells under conditions which are permissive for infection of a third fraction of cells in the cell population with the adenovirus, wherein the third fraction of cells is infected by adenovirus released by the first and/or second fraction of adenovirus-infected cells.

In preferred embodiments, the methods of the invention comprise culturing a cell population comprising a first fraction of adenovirus-infected cells under conditions which are permissive for infection of a second fraction of cells in the cell population with the adenovirus, wherein the second fraction of cells is infected by adenovirus released into the culture by the first fraction of infected cells. In some embodiments, prior to culturing the cell population comprising a first fraction of adenovirus-infected cells under conditions which are permissive for infection of a second fraction of the cell population with the adenovirus, the method comprises culturing the cell population under conditions which are permissive for infection of the first fraction of cells in the cell population with the adenovirus. In some embodiments, prior to culturing the cell population under conditions which are permissive for infection of the first fraction of cells in the cell population with the adenovirus, the method comprises adding the adenovirus to the cell population in culture.

In some embodiments of the methods of the invention, the conditions which are permissive for infection of the cell population with adenovirus comprise adding a cell culture additive to the cell population. As shown in Example 5, such additives may increase the viral titer. Accordingly, in some embodiments, the methods of the invention comprise adding a cell culture additive to the cell population. In some embodiments, the conditions which are permissive for infection of the cell population with adenovirus comprise culturing the cell population in the presence of a cell culture additive as defined herein. In some embodiments, the method comprises adding the cell culture additive to the cell population while culturing the cell population under conditions which are permissive for infection of the cell population. As used herein a “cell culture additive” means a cell culture additive which is not present during the initial cell seeding step.

In some embodiments, the cell culture additive comprises DMSO. In some embodiments, the cell culture additive comprises sodium butyrate. In some embodiments, the cell culture additive comprises CaCl₂. In preferred embodiments, the cell culture additive comprises DMSO, sodium butyrate, and/or CaCl₂. In particularly preferred embodiments, the cell culture additive comprises DMSO, sodium butyrate, and CaCl₂. In some embodiments, after adding the cell culture additive to the cell population, the cell population is exposed to from about 0.1% to about 4% DMSO, preferably from about 0.5% to about 2% DMSO, most preferably about 0.5% or about 1% DMSO. In some embodiments, after adding the cell culture additive to the cell population, the cell population is exposed to from about to about 10 mM sodium butyrate, preferably from about 0.5 mM to about 2.5 mM sodium butyrate, most preferably about 1 mM sodium butyrate. In some embodiments, after adding the cell culture additive to the cell population, the cell population is exposed to from about 0.5 mM to about 10 mM CaCl₂, preferably from about 1 mM to about 5 mM CaCl₂, most preferably about 2 mM CaCl₂.

The cell culture additive may be added to the cell population at various time points. For example, in some embodiments, the methods of the invention comprise adding the cell culture additive to the cell population about 0-148 hours after adding the adenovirus to the cell population, preferably about 48-120 hours after adding the adenovirus to the cell population, most preferably about 72-120 hours after adding the adenovirus to the cell population. In some embodiments, the method comprises adding a cell culture additive to the cell population approximately at least every 12-96 hours, preferably approximately at least every 24-72 hours, most preferably approximately every 48 hours.

In some embodiments of the methods of the invention, the conditions which are permissive for infection of the cell population with adenovirus comprise adding a feed to the cell population. Accordingly, in some embodiments, the methods of the invention comprise adding a feed to the cell population. In some embodiments, the conditions which are permissive for infection of the cell population with adenovirus comprise culturing the cell population in the presence of a feed as defined herein. In some embodiments, the method comprises adding the feed to the cell population while culturing the cell population under conditions which are permissive for infection of the cell population. As used herein a “feed” means a cell culture nutrient (such as amino acids and/or glucose) which is not present during the initial cell seeding step. Accordingly, in some embodiments, the feed comprises amino acids, vitamins and/or glucose. In preferred embodiments, the feed comprises amino acids, vitamins and glucose. In some embodiments, the feed is BalanCD® HEK293 Feed.

The feed may be added to the cell population at various time points. For example, in some embodiments, the method comprises adding the feed to the cell population about 0-120 hours after adding the adenovirus to the cell population, preferably about 24-96 hours after adding the adenovirus to the cell population, most preferably about 24-48 hours after adding the adenovirus to the cell population. In some embodiments, the method comprises adding feed to the cell population approximately at least every 12-96 hours, preferably approximately at least every 24-72 hours, most preferably approximately every 48 hours. In some embodiments, the method comprises adding the feed to the cell population at a final concentration of up to about 10% v/v, preferably up to about 7.5% v/v, most preferably at a final concentration of about 5% v/v. In preferred embodiments, the method comprises adding the feed to the cell population about 24-48 hours after adding the adenovirus to the cell population at a final concentration of about 5% v/v.

In some embodiments, the conditions which are permissive for infection of the cell population with adenovirus are conditions which maintain cell viability >80%, preferably >85%, most preferably >90%. In some embodiments of the methods of the invention, the cell population is cultured under conditions which maintain cell viability >80%, preferably >85%, most preferably >90%. Cell viability can be determined by a number of techniques known in the art. For example, the dye exclusion technique utilizes an indicator dye to identify cell membrane damage. Cells which absorb the dye become stained and are considered non-viable. Dyes such as trypan blue, erythrosine, and nigrosine are commonly used. Cell viability may be calculated using an automated machine, such as a Vi-CELL™ XR Cell Viability Analyzer.

In some embodiments, the conditions which are permissive for infection of the cell population with adenovirus comprise agitating the cell population. Accordingly, in some embodiments of the methods of the invention, the method comprises agitating the cell population. For example, in some embodiments, the cell population is cultured in a cell culture vessel (e.g. bioreactor) set to have an agitation rate that results in power input from about 1 to about 100 W/m³, for example from about 5 to about 90 W/m³, preferably from about 15 to about 70 W/m³.

The inventors have surprisingly found that exposing the cell population to a first and second temperature, wherein the first temperature is higher than the second temperature, can result in the production of a higher yield of adenovirus (see Example 6). As used herein, “first temperature” refers to a temperature at which the cell population is cultured prior to addition of adenovirus to the cell population, for example about 31-40° C., preferably about 35-38° C., most preferably about 37° C., and “second temperature” is a temperature that is different from (e.g. lower than) the first temperature, for example about 27-40° C., preferably about 31-35° C., most preferably about 33° C. In some embodiments, the second temperature is about 1-10° C. lower than the first temperature, preferably about 3-7° C. lower than the first temperature, most preferably about 4° C. lower than the first temperature.

Accordingly, in some embodiments of the methods of the invention, the method comprises culturing the cell population at a first temperature. In some embodiments, the conditions which are permissive for infection of the cell population comprise culturing the cell population at a first temperature. For example, the conditions which are permissive for infection of the cell population may comprise culturing the cell population at a first temperature of about 31-40° C., preferably about 35-38° C., and most preferably about 37° C. In some embodiments, the method comprises culturing the cell population at a second temperature. In some embodiments, the conditions which are permissive for infection of the cell population comprise culturing the cell population at a second temperature, i.e. a temperature that is different from (e.g. lower than) the first temperature. For example, the conditions which are permissive for infection of the cell population may comprise culturing the cell population at a second temperature of about 31-40° C., preferably about 31-35° C., most preferably about 33° C. In preferred embodiments, the conditions which are permissive for infection of the cell population with the adenovirus comprise culturing the cell population at the first temperature followed by culturing the cell population at the second temperature.

In some embodiments, the conditions which are permissive for infection of the first fraction of cells in the cell population comprise culturing the cell population at a first temperature. For example, in some embodiments, the conditions which are permissive for infection of the first fraction of cells comprise culturing the cell population at a first temperature of about 31-40° C., preferably about 35-38° C., and most preferably about 37° C. In some embodiments, the conditions which are permissive for infection of the second fraction of cells in the cell population comprise culturing the cell population at the first temperature. In some embodiments, the conditions which are permissive for infection of the second fraction of cells in the cell population comprise culturing the cell population at a second temperature, i.e. a temperature that is different from (e.g. lower than) the first temperature. For example, in some embodiments, the conditions which are permissive for infection of the second fraction of cells comprise culturing the cell population at a second temperature of about 27-40° C., preferably about 31-35° C., and most preferably about 33° C. In preferred embodiments, the conditions which are permissive for infection of the first fraction of cells comprise culturing the cell population at the first temperature, and the conditions which are permissive for infection of the second fraction of cells comprise culturing the cell population comprising the first fraction of infected cells at the second temperature.

After infection of a cell, adenovirus is transported to the nucleus of the cell. The viral DNA is then released allowing it to enter the nucleus of the cell and replicate. In some embodiments, the methods of the invention comprise culturing the cell population comprising adenovirus-infected cells under conditions which are permissive for replication of the adenovirus. As used herein, “conditions which are permissive for replication of the adenovirus” means any suitable conditions permitting propagation of the adenovirus within the cells. Such conditions may be dependent on the cell type being cultured and the adenovirus used to infect the cells. In preferred embodiments, the pH of the culture is maintained at about 6.5-7.5, more preferably at about 6.9-7.3. Preferably, pH and/or other conditions will be maintained to optimise glucose metabolism by the cells. The pH of a cell culture can be controlled by any suitable method, preferably in a manner that does not substantially inhibit the production of the adenovirus. Several suitable techniques for modifying pH are known in the art, including the addition of buffers (e.g., bicarbonate or tris buffers). Proper mixing of the culture is another condition which can be important to cell growth and adenovirus production. Other factors which may be considered include temperature, agitation rate, oxygen concentration, CO 2 perfusion rate, concentration of cells, settling and flow rates of cells in the culture, and levels of particular nutrients and/or intermediates that impact cell growth and metabolism rates (e.g. glutamine). Techniques for determining propagation of the adenovirus within the cells are well known in the art and include qPCR to determine gene copy number, plaque assays to determine infectious virus titer and HPLC to determine viral particle number.

In some embodiments, the conditions which are permissive for replication of the adenovirus comprise adding a cell culture additive as defined herein to the cell population. In some embodiments, the conditions which are permissive for replication of the adenovirus comprises culturing the cell population in the presence of a cell culture additive. In some embodiments, the method comprises adding the cell culture additive to the cell population while culturing the cell population comprising adenovirus-infected cells under conditions which are permissive for replication of the adenovirus.

In some embodiments, the conditions which are permissive for replication of the adenovirus comprise adding a feed as defined herein to the cell population. In preferred embodiments, the conditions which are permissive for replication of the adenovirus comprises culturing the cell population in the presence of a feed. In some embodiments, the method comprises adding the feed to the cell population while culturing the cell population comprising adenovirus-infected cells under conditions which are permissive for replication of the adenovirus.

In some embodiments, the conditions which are permissive for replication of the adenovirus are conditions which maintain cell viability >80%, preferably >85%, most preferably >90%. In preferred embodiments, the conditions which are permissive for infection of the cell population with the adenovirus and the conditions which are permissive for replication of the adenovirus are conditions which maintain cell viability >80%, preferably >85%, most preferably >90%.

In some embodiments, the conditions which are permissive for replication of the adenovirus comprise agitating the cell population. For example, in some embodiments, the cell population is cultured in a vessel (e.g. bioreactor) set to have an agitation rate that results in power input from about 1 to about 100 W/m³, for example from about 5 to about 90 W/m³, preferably from about 15 to about 70 W/m³.

In some embodiments, the conditions which are permissive for replication of the adenovirus comprise culturing the cell population at a first temperature as defined herein. In preferred embodiments, the conditions which are permissive for replication of the adenovirus comprise culturing the cell population at a second temperature as defined herein. In preferred embodiments, the conditions which are permissive for infection of the cell population with the adenovirus comprise culturing the cell population at a first temperature defined herein and the conditions which are permissive for replication of the adenovirus comprise culturing the cell population at a second temperature defined herein.

Accordingly, in some embodiments, the methods of the invention comprise switching the temperature to which the cell population is exposed from a first temperature to a second temperature. In preferred embodiments, the first temperature is permissive for infection of the cell population with the adenovirus. In some embodiments, the second temperature is permissive for infection of the cell population with the adenovirus. In preferred embodiments, the first and second temperatures are permissive for infection of the cell population with the adenovirus. In some embodiments, the first temperature is permissive for replication of the adenovirus. In preferred embodiments, the second temperature is permissive for replication of the adenovirus. In preferred embodiments, the first and second temperatures are permissive for replication of the adenovirus. In preferred embodiments, the first temperature is permissive for infection of the cell population with the adenovirus and the second temperature is permissive for replication of the adenovirus. In preferred embodiments, the first and second temperatures are permissive for infection of the cell population with the adenovirus and the first and second temperatures are permissive for replication of the adenovirus.

In some embodiments, the methods of the invention comprise switching the temperature to which the cell population is exposed from the first temperature to the second temperature about 3-96 hours after adding the adenovirus to the cell population, preferably about 48-96 hours after adding the adenovirus to the cell population, most preferably about 72 hours after adding the adenovirus to the cell population. In some embodiments, the cell population is cultured at the first temperature for at least about 24 hours, preferably at least about 72 hours, most preferably for at least about 96 hours. In some embodiments, the cell population is cultured at the second temperature for at least about 24 hours, preferably at least about 36 hours, most preferably at least about 48 hours. In some embodiments, after switching the temperature from the first temperature to the second temperature, the cell population is cultured at the second temperature until the adenovirus is harvested from the culture.

In some embodiments of the methods of the invention, exposing the cell population to the second temperature increases the stability of the adenovirus in the culture. For example, exposing the cell population to the second temperature may increase the stability of the adenovirus in the culture compared with merely exposing the cell population to the first temperature. Adenovirus stability can readily be determined by any suitable method, for example by measuring viral genome titer (e.g. by qPCR) or infectious titer (e.g. by plaque assay) over time. If the virus is stable, the virus titer is not expected to decrease. Conversely, if the virus is not stable, the virus titer is expected to decrease. In some embodiments, exposing the cell population to the second temperature decreases the adenovirus particle:infectious particle ratio in the culture. For example, exposing the cell population to the second temperature may decrease the adenovirus particle:infectious particle ratio in the culture compared with merely exposing the cell population to the first temperature. Determination of the adenovirus particle:infectious particle ratio may be assessed by any suitable method, for example by measuring the viral particle titer and infectious titer separately, and then taking the ratio of the two values. In preferred embodiments, exposing the cell population to the second temperature increases the stability of the adenovirus in the culture and decreases the adenovirus particle:infectious particle ratio in the culture.

Advantageously, the number of viable cells in the cell population may increase after addition of adenovirus to the cell population (see Examples 1, 2 and 7). In some embodiments of the methods of the invention, the first temperature is permissive for growth of the cell population. In some embodiments, the second temperature is permissive for growth of the cell population. In some embodiments, the first and second temperatures are permissive for growth of the cell population. Accordingly, in some embodiments, the methods of the invention comprise culturing the cell population under conditions which are permissive for growth of the cell population. As used herein, “permissive for growth of the cell population” means any suitable manner of culturing the cell population that permits the growth of cells. The method of culturing such cells will depend upon the cell type selected. Suitable culturing methods are well known in the art, and typically involve maintaining pH and temperature within ranges suitable for growth of the cells. Preferred temperatures for culturing are about 27-40° C., more preferably 31-37° C., and optimally about 37° C. Preferably, the pH of the culture is maintained at about 6-8, more preferably at about 6.7-7.8, and optimally at about 6.9-7.5. Cell density may increase throughout the growth cycle of the cell population. The concentration of the cells in the culture can be monitored throughout the process using numerous techniques well known in the art. Techniques focusing on total number of cells in the culture include determining the weight of the culture, assessing culture turbidity, determining metabolic activity in the culture, electronic cell counting, microscopic cell counting of culture samples, plate counting using serial dilutions, membrane filter counting, and radioisotope assays. In the present invention, any technique permissive for assessing cell density is suitable. For example. cell density of a culture can be determined spectrophotometrically or by using a counting chamber, such as hemocytometer. Cell density may be calculated using an automated machine such as a Vi-CELL™ XR Cell Viability Analyzer.

In some embodiments, the conditions which are permissive for infection of the cell population are conditions permissive for cell growth. In some embodiments, the conditions which are permissive for infection of the first fraction of the cell population are conditions permissive for cell growth. In some embodiments, the conditions which are permissive for infection of the second fraction of the cell population are conditions permissive for cell growth. In some embodiments, the conditions which are permissive for infection of the third fraction of the cell population are conditions permissive for cell growth. In some embodiments, the conditions which are permissive for replication of the adenovirus are conditions permissive for cell growth.

After the initial cell infection, an adenovirus may undergo several rounds of infection of the cell population and/or replication in a cell. Accordingly, in some embodiments of the methods of the invention, peak virus titer is achieved about 2-8 days after adding the adenovirus to the cell population, preferably about 3-7 days after adding the adenovirus to the cell population, most preferably about 4-6 days after adding the adenovirus to the cell population.

After replication, the adenovirus may advantageously by isolated from the culture (e.g. for purification purposes so that the adenovirus can be added to a vaccine). Accordingly, in some embodiments, the methods of the invention comprise harvesting the adenovirus from the culture. In some embodiments, the step of harvesting the adenovirus from the culture comprises harvesting adenovirus from the cell culture medium in which the cell population was cultured. In preferred embodiments, the step of harvesting the adenovirus from the culture comprises lysing the cells of the cell population. In some embodiments, the step of harvesting the adenovirus from the culture comprises lysing the cells of the cell population and harvesting adenovirus from the cell lysate of the cell population.

In preferred embodiments, the cells of the cell population are lysed using a cell lysis agent (e.g. a detergent). Use of a detergent for cell lysis has the advantage that it is straightforward to implement, and that it is easily scalable. Detergents that can be used for cell lysis are known in the art. Detergents used for cell lysis in the methods of the present invention can include but are not limited to anionic, cationic, zwitterionic, and nonionic detergents. In preferred embodiments, the detergent is a nonionic detergent. Examples of suitable nonionic detergents include Polysorbate (e.g. Polysorbate-20 or Polysorbate-80) and Triton (e.g. Triton-X). In one embodiment, the nonionic detergent is Polysorbate-20. The optimal concentration of the nonionic detergent used to lyse the host cell population may vary, for instance within the range of about 0.005-0.025 kg detergent/kg cell culture vessel, about 0.01-0.02 kg detergent/kg cell culture vessel, or about 0.011-0.016 kg detergent/kg cell culture vessel. As used herein, “kg cell culture vessel” means the total mass of the cell population and the cell culture medium in the cell culture vessel. In preferred embodiments, the concentration of the nonionic detergent (e.g. Polysorbate-20) used to lyse the host cell population is about 0.013 kg detergent/kg cell culture vessel. The host cells may be incubated with the nonionic detergent (e.g. Polysorbate-20) for sufficient time for all or substantially all of the cells in the host cell population to be lysed. In embodiments, the host cells are incubated with the nonionic detergent (e.g. Polysorbate-20) for at least about 15 minutes prior to a nuclease treatment step. In embodiments, the host cells are incubated with the nonionic detergent (e.g. Polysorbate-20) for up to 30 minutes prior to a nuclease treatment step. In embodiments, the host cells are not incubated with the nonionic detergent (e.g. Polysorbate-20) for longer than 30 minutes prior to a nuclease treatment step.

In embodiments, the detergent (e.g. Polysorbate-20) forms part of a lysis buffer. Accordingly, in some embodiments, the host cells are lysed using a lysis buffer comprising at least one detergent (e.g. Polysorbate-20). An exemplary lysis buffer that may be used in the methods of the invention comprises about 500 mM tris, about 20 mM MgCl₂, about 50% (w/v) sucrose and about 10% (v/v) Polysorbate 20, and has a pH of about 8. The optimal concentration of the lysis buffer used to lyse the cell population may vary, for instance within the range of about 0.05-0.25 kg lysis buffer/kg vessel, about 0.10-0.20 kg lysis buffer/kg vessel, or about 0.11-0.16 kg lysis buffer/kg vessel. In preferred embodiments, the concentration of the lysis buffer is about 0.13 kg lysis buffer/kg vessel.

In some embodiments, the step of harvesting the adenovirus from the culture is performed at least about 48 hours after adding the adenovirus to the cell population, preferably at least about 96 hours after adding the adenovirus to the cell population, most preferably at least about 120 hours after adding the adenovirus to the cell population. For example, in some embodiments the step of harvesting adenovirus from the culture is performed about 96-144 hours after adding the adenovirus to the cell population.

In some embodiments, the step of harvesting the adenovirus from the culture is performed when the viability of the cells in the cell population decreases, for example when the viability of the cells in the cell population decreases below about 99%, below about 97%, below about 95%, below about 90% or below about 80%, preferably when the viability of the cell population decreases below about 95%. For example, in some embodiments the step or harvesting the adenovirus from the culture is performed when fewer than about 99%, about 97%, about 95%, about 90% or about 80%, preferably when fewer than about 95% of the cells in the cell population are viable. In some embodiments, the step of harvesting the adenovirus from the cell culture is performed when the oxygen consumption of the cells decreases. Accordingly, in some embodiments, the step of harvesting the adenovirus from the culture is performed when the viable cell density decreases below about 1.5×10⁷ cells/mL, below about 1×10⁷ cells/mL, below about 6×10⁶ cells/mL, below about 5.5×10⁶ cells/mL, below about 5×10⁶ cells/mL or below about 4×10⁶ cells/mL.

In some embodiments, the host cell population may have a cell density (e.g. viable cell density) at time of harvest of at least about 6×10⁵ cells/mL, at least about 8×10⁵ cells/mL, at least about 1×10⁶ cells/mL, at least about 2×10⁶ cells, at least about 4×10⁶ cells, at least about 6×10⁶ cells, at least about 8×10⁶ cells or at least about 1×10⁷ cells. In preferred embodiments, the host cell population has a cell density (e.g. viable cell density) at time of harvest of at least about 4×10⁶ cells. In some embodiments, the step of harvesting the adenovirus from the cell culture is performed when the oxygen consumption of the cells decreases.

The host cell population may have a cell density (e.g. viable cell density) at time of harvest of up to about 1×10⁹ cells/mL, up to about 1×10⁸ cells/mL, up to about 8×10⁷ cells/mL, up to about 6×10⁷ cells/mL, up to about 4×10⁷ cells/mL, up to about 2×10⁷ cells/mL, up to about 1×10⁷ cells/mL, up to about 8×10⁶ cells/mL or up to about 6×10⁶ cells/mL. In some embodiments, the host cell population has a cell density (e.g. viable cell density) at time of harvest of up to about 8×10⁶ cells/mL.

The host cell population may have a cell density (e.g. viable cell density) at time of harvest of between about 1×10⁵ cells/mL and about 1×10⁹ cells/mL, between about 8×10⁵ cells/mL and about 1×10⁸ cells/mL or between about 1×10⁶ cells/mL and about 1×10⁷ cells/mL.

At the time of harvest, the adenovirus-containing culture may comprise at least one host cell protein (HCP). As used herein, the term “HCP” refers to proteins produced or encoded by a host cell population.

The adenovirus-containing culture may have a HCP concentration of at least about 20,000 ng/mL, at least about 30,000 ng/mL, at least about 40,000 ng/mL, at least about 50,000 ng/mL, at least about 60,000 ng/mL, at least about 70,000 ng/mL, at least about 80,000 ng/mL, at least about 90,000 ng/mL or at least about 100,000 ng/mL. In preferred embodiments, the adenovirus-containing culture has a HCP concentration of at least about 50,000 ng/mL.

The adenovirus-containing culture may have a HCP concentration of up to about 100,000 ng/mL, up to about 90,000 ng/mL, up to about 80,000 ng/mL, up to about 70,000 ng/mL, up to about 60,000 ng/mL, up to about 50,000 ng/mL, up to about 40,000 ng/mL, up to about 30,000 ng/mL, or up to about 20,000 ng/mL. In preferred embodiments, the adenovirus-containing culture has a HCP concentration of up to about 75,000 ng/mL.

The adenovirus-containing culture may have a HCP concentration of between about 20,000 ng/mL and about 100,000 ng/mL, between about 30,000 ng/mL and about 90,000 ng/mL or between about 50,000 ng/mL and about 80,000 ng/mL.

In preferred embodiments, the host cell population has a cell density (e.g. viable cell density) at time of harvest and having a HCP concentration as set forth above. For example, the host cell population may have a cell density of at least about 4×10⁶ cells and a HCP concentration of at least about 50,000 ng/mL.

In some embodiments of the methods of the invention, the step of harvesting the adenovirus from the cell culture is performed when the viral titer is above a threshold. For example, in some embodiments, the viral titer at the time of harvesting is at least about 0.5×10¹⁰ GC/mL, preferably at least about 1×10¹¹ GC/mL, at least about 2×10¹¹ GC/mL, at least about 3×10¹¹ GC/mL, or at least about 4×10¹¹ GC/mL, most preferably when the viral titer is at least about 2×10¹¹ GC/mL.

During production of the adenovirus, the cells in culture may need to be provided with fresh nutrients to allow the cells to remain viable. Accordingly, in some embodiments, the methods of the invention comprise a step of replacing or adding cell culture medium to the cell population after adding the adenovirus to the cell population. However, replacement or addition of cell culture medium is costly and time consuming. Therefore, in preferred embodiments, the method does not comprise a step of replacing or adding cell culture medium to the cell population after adding the adenovirus to the cell population.

The present inventors have shown that the methods of the present invention can be performed on a large scale (see e.g. Example 7). For example, the methods of the present invention may be suitable for harvesting up to about 5000 litres, e.g. from about 3 litres to about 3000 litres, preferably in the range of about 200 litres to about 2000 litres of adenovirus-containing material (e.g. cell lysate and/or cell culture medium, preferably cell lysate) from a single culture. Accordingly, in some embodiments of the methods of the invention, the culture process takes place in a bioreactor. As used herein “bioreactor” means a cell culture vessel adapted for a large scale process. For example, in some embodiments, the bioreactor has a capacity of at least about 1 L, preferably at least about 1.2 L, about 3 L, about 50 L, about 1000 L, about 2000 L, about 3000 L, or about 5000 L, most preferably at least about 2000 L. In some embodiments, the bioreactor has a capacity of at least about 7×10⁹ viable T-REx™ cells, preferably at least about 2.1×10¹⁰ viable T-REx™ cells, at least about 3.5×10¹¹ viable T-REx™ cells, at least about 5×10¹² viable T-REx™ cells or at least about 3×10¹³ viable T-REx™ cells, most preferably at least about 5×10¹² viable T-REx™ cells.

Adenovirus Vectors

In preferred embodiments of the methods of the invention, the adenovirus is an adenovirus vector. As used herein “adenovirus vector” means a form of an adenovirus which has been modified for insertion of a nucleotide sequence encoding a heterologous gene into a eukaryotic cell. As used herein, “heterologous gene” means a gene derived from a genotypically distinct entity from that of the rest of the entity to which it is being compared. Thus, a heterologous gene refers to any gene that is not isolated from, derived from, or based upon a naturally occurring gene of the adenovirus. As used herein “naturally occurring” means found in nature and not synthetically prepared or modified.

In preferred embodiments of the methods of the invention, the adenovirus vector comprises a heterologous gene encoding a protein of interest, for example a therapeutic protein or an immunogenic protein. Alternatively, a heterologous gene may include a reporter gene, which upon expression produces a detectable signal. Such reporter genes include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry.

In preferred embodiments, the heterologous gene is a sequence encoding a product, such as protein, RNA, enzyme or catalytic RNA, which is useful in biology and medicine, such as a therapeutic gene or an immunogenic gene. The heterologous gene may be used for treatment, e.g. of genetic deficiencies, as a cancer therapeutic, as a vaccine, for induction of an immune response, and/or for prophylactic purposes. In preferred embodiments, the heterologous gene encodes a foreign antigen such as a naturally occurring form of a foreign antigen, or a modified form thereof. As used herein, “foreign antigen” means an antigen which induces a host immune response and is derived from a genotypically distinct entity from that of the host in which it induces the immune response. As used herein, a modified form of a foreign antigen means a form of the foreign antigen which induces a host immune response against the naturally occurring antigen and has at least 50%, at least 60%, at least 70%, at least 80%, at least 85%, at least 90%, at least 95%, at least 98%, or at least 99% sequence identity to the naturally occurring antigen. As used herein, induction of an immune response refers to the ability of a protein to induce a T cell and/or a humoral immune response to the protein. Determination of a host immune response against a naturally occurring form of a foreign antigen, or a modified form thereof, may be assessed by any suitable method such as those described in Jeyanathan et al. 2020; Immunological considerations for COVID-19 vaccine strategies; Nature Reviews Immunology 20, 615-632 and Albert-Vega et al. 2018; Immune Functional Assays, From Custom to Standardized Tests for Precision Medicine; Frontiers in Immunology 9:2367. In some embodiments, the modified form of the naturally occurring antigen induces a more powerful host immune response than that induced by the naturally occurring antigen. In some embodiments, the modified form of the naturally occurring antigen induces a weaker host immune response than that induced by the naturally occurring antigen.

In some embodiments, the foreign antigen is derived from SARS-CoV2, preferably from the spike protein of SARS-CoV2. SARS-CoV2 is a newly-emergent coronavirus which causes a severe acute respiratory disease, COVID-19. Thus far, no vaccine has been available on a global scale to prevent SARS-CoV2 infection. Because this virus uses its spike glycoprotein for interaction with the cellular receptor ACE2 and the serine protease TMPRSS2 for entry into a target cell, this spike protein represents an attractive target for vaccine therapeutics. Accordingly, in preferred embodiments, the heterologous gene codes for a naturally occurring form of the SARS-CoV2 spike protein, or a modified version thereof. The RNA, DNA, and amino acid sequence of the SARS-CoV2 spike protein are known to those skilled in the art and can be found in many databases, for example, in the database of the National Center for Biotechnology Information (NCBI), where it has an accession number of NC 045512.2. For example, in some embodiments, the heterologous gene encodes the SARS-CoV2 spike protein comprising an amino acid sequence set forth in SEQ ID NO: 1. In other exemplary embodiments, the heterologous gene encodes a modified form of the SARS-CoV2 spike protein comprising an amino acid sequence set forth in SEQ ID NO: 2. As will be readily understood, the amino acid sequence set forth in SEQ ID NO: 2 comprises the SARS-CoV2 spike protein amino acid sequence with the signal peptide of the human tissue plasminogen activator gene (tPA) at the N terminus. Presence of the N-terminal tPA sequence may enhance immunogenicity of the SARS-CoV2 spike protein.

In addition to the heterologous gene, the vector may also include conventional control elements which are operably linked to the heterologous gene in a manner that permits its transcription, translation and/or expression in a cell infected with the adenovirus. As used herein “operably linked” includes both expression control sequences that are contiguous with the gene of interest and expression control sequences that act in trans or at a distance to control the gene of interest.

Expression control sequences may include appropriate transcription initiation, termination, promoter and enhancer sequences; efficient RNA processing signals such as splicing and polyadenylation (poly A) signals; sequences that enhance translation efficiency; sequences that enhance protein stability; and when desired, sequences that enhance secretion of the encoded product. As used herein, a “promoter” is a nucleotide sequence that permits binding of RNA polymerase and directs the transcription of a gene. A number of expression control sequences, including promoters which are internal, native, constitutive, inducible and/or tissue-specific are known in the art and may be utilized.

The adenovirus vector may be derived from a mammalian adenovirus. In some embodiments of the methods of the invention, the adenovirus vector is derived from a human adenovirus. In some embodiments, the human adenovirus is a serotype 5 human adenovirus. In preferred embodiments, the human adenovirus is not a serotype 5 human adenovirus.

In preferred embodiments, the adenovirus vector is not derived from a human adenovirus. Thus, the adenovirus vector may be derived from a non-human adenovirus, for example, a chimpanzee adenovirus. In particularly preferred embodiments, the adenovirus vector is derived from a chimpanzee adenovirus, e.g. ChAdOx1 (Antrobus et al. 2014 Mol. Ther. 22(3):668-674), ChAdOx2 (Morris et al. 2016 Future Virol. 11(9):649-659), ChAd3 or ChAd63. In especially preferred embodiments, the adenovirus vector is derived from ChAdOx1.

In some embodiments of the methods of the invention, the adenovirus vector is for use in a vaccine and is derived from the same species as the species for which the vaccine is targeted. For example, in some embodiments, the vaccine is targeted to a disease found in humans and the adenovirus vector is derived from a human adenovirus. In preferred embodiments, however, the adenovirus vector is for use in a vaccine and is derived from a species different to that for which the vaccine is targeted. For example, in some embodiments, the vaccine is targeted to a disease found in humans and the adenovirus vector is derived from a non-human adenovirus, such as a chimpanzee adenovirus. It is thought that the use of an adenovirus vector derived from a species different from the species for which a vaccine is targeted may provide an improved vaccine that encounters a lower incidence of pre-existing anti-adenoviral immunity when administered.

Adenovirus vectors may be engineered so that they are unable to replicate after administration to a host. Accordingly. in some embodiments of the methods of the invention, the adenovirus vector is a replication deficient adenovirus vector (e.g. replication deficient adenovirus vector derived from chimpanzee adenovirus). As used herein, a “replication deficient adenovirus vector” means an adenovirus vector which is unable to replicate in a host cell lacking one or more adenovirus replication genes. In some embodiments, the adenovirus vector lacks an E1A gene. In some embodiments, the adenovirus vector has been modified to prevent elimination of cells infected with the adenovirus vector by the host immune system. For example, in some embodiments, the adenovirus vector lacks an E1B gene and/or an E3 gene. In some embodiments, the adenovirus vector lacks an E1B gene. In some embodiments, the adenovirus vector lacks an E3 gene. In some embodiments, the adenovirus vector lacks an E1B gene and an E3 gene. In some embodiments, the adenovirus vector is a minimal adenovirus vector comprising an origin of replication (ori) and a packaging sequence. In some embodiments, the minimal adenovirus vector further comprises a heterologous gene encoding a protein of interest.

Cell Population

In preferred embodiments of the methods of the invention, the cell population is complementary to the adenovirus added to the cell population. As used herein, a “cell population complementary to an adenovirus being produced” is a cell population which has been engineered to express an adenovirus factor which is not expressed by the adenovirus being produced. For example, in some embodiments, the adenovirus added to the cell population does not express an adenovirus DNA replication factor and the cell population expresses the adenovirus DNA replication factor. As used herein an “adenovirus DNA replication factor” is a factor which in nature, forms part of the adenovirus DNA, and is required for the adenovirus to replicate in a host cell. Accordingly, in some embodiments, the adenovirus added to the cell population does not express an E1A protein, an E1B protein, and/or an E4 protein and the cell population expresses the E1A protein, the E1B protein, and/or the E4 protein.

The cell population may be a primary cell population which has been freshly isolated from a tissue. In some embodiments, the tissue is a mammalian tissue.

Alternatively, the cell population may be derived from a cell line which has been adapted for culture. In some embodiments, the cell line is an immortalised cell line. In some embodiments, the cell line is a mammalian cell line. In some embodiments, the cell population comprises mammalian cells. For example, in some embodiments the cell population comprises human embryonic kidney (HEK) cells or is a HEK cell line. The mammalian cells may express an adenovirus replication factor. For example, in some embodiments, the cell population expresses an E1A protein, an E1B protein, and/or an E4 protein. In some embodiments, the cell population expresses a tetracycline repressor protein. In preferred embodiments, the cell population comprises T-REx™ cells. In some embodiments, the cell population consists of T-REx™ cells. In a preferred embodiment, the cell population comprises Expi293F inducible cells (Thermofisher) or modified T-REX™ cells.

Vaccine Production

Adenoviruses comprising a heterologous gene may be administered in immunogenic compositions. As used herein an “immunogenic composition” is a composition comprising an adenovirus produced according to a methods of the invention which is capable of inducing an immune response, for example a humoral (e.g. antibody) and/or cell-mediated (e.g. cytotoxic T cell) response, against the heterologous gene product delivered by the vector following delivery to a mammal, preferably a human. Thus, an adenovirus produced according to the invention may comprise a gene encoding a desired immunogen and may therefore be used in a vaccine. The adenoviruses can be used as prophylactic or therapeutic vaccines against any pathogen for which the antigen(s) crucial for induction of an immune response and able to limit the spread of the pathogen has been identified and for which the cDNA is available.

Accordingly, in one aspect there is provided a method for making a vaccine, the method comprising producing an adenovirus according to a method of the invention, purifying the adenovirus, and preparing a vaccine comprising the purified adenovirus. Methods of purifying an adenovirus for use in a vaccine are well known in the art, for example, as described in Vellinga et al. 2014; Challenges in Manufacturing Adenoviral Vectors for Global Vaccine Product Development; Human Gene Therapy 25:318-327.

Such vaccine or other immunogenic compositions may be formulated in a suitable delivery vehicle. The levels of immunity to the heterologous gene encoded by the adenovirus can be monitored to determine the need, if any, for boosters. Following an assessment of antibody titers in the serum, optional booster immunizations may be desired.

In some embodiments, the vaccine comprises an adjuvant. As used herein, an “adjuvant” means a composition that enhances the immune response to an immunogen. Examples of adjuvants include but are not limited to inorganic adjuvants (e.g. inorganic metal salts such as aluminium phosphate or aluminium hydroxide), organic adjuvants (e.g. saponins, such as QS21, or squalene), oil-based adjuvants (e.g. Freund's complete adjuvant and Freund's incomplete adjuvant), cytokines (e.g. IL-1β, IL-2, IL-7, IL-12, IL-18, GM-CFS, and IFN-γ) particular adjuvants (e.g. immuno-stimulatory complexes (ISCOMS), liposomes, or biodegradable microspheres), virosomes, bacterial adjuvants (e.g. monophosphoryl lipid A, such as 3-de-O-acylated monophosphoryl lipid A (3D-MPL), or muramyl peptides), synthetic adjuvants (e.g. non-ionic block copolymers, muramyl peptid analogues, or synthetic lipid A), synthetic polynucleotides adjuvants (e.g. polyarginine or polylysine) and immunostimulatory oligonucleotides containing unmethylated CpG dinucleotides (“CpG”).

In some embodiments, the adjuvant is formulated together with carriers, such as liposomes, oil in water emulsions, and/or metallic salts.

In preferred embodiments of the methods of the invention, the vaccine is a COVID-19 vaccine.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure. In addition, it will be understood that any of the embodiments described herein are applicable to any of the aspects described herein.

Concentrations, amounts, volumes, percentages and other numerical values may be presented herein in a range format. Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

It must be noted that as used herein and in the appended claims, the singular forms “a”, “an”, and “the” include plural referents unless the context clearly dictates otherwise. Thus, for example, reference to “an agent” includes a plurality of such agents and reference to “the agent” includes reference to one or more agents and equivalents thereof known to those skilled in the art, and so forth.

“About” may generally mean an acceptable degree of error for the quantity measured given the nature or precision of the measurements. Exemplary degrees of error are within percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

Embodiments described herein as “comprising” one or more features may also be considered as disclosure of the corresponding embodiments “consisting essentially of” such features, or “consisting of” such features.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

All documents cited herein are each entirely incorporated by reference herein, including all data, tables, figures, and text presented in the cited documents.

### EXAMPLES

### Example 1—Adenovirus Infection Using High and Low MOI

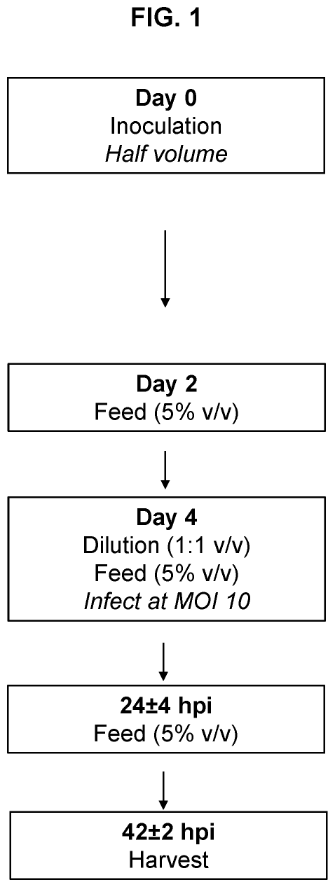

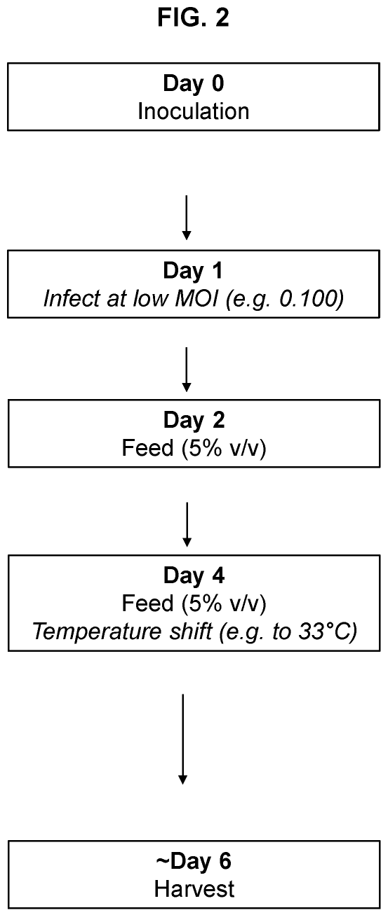

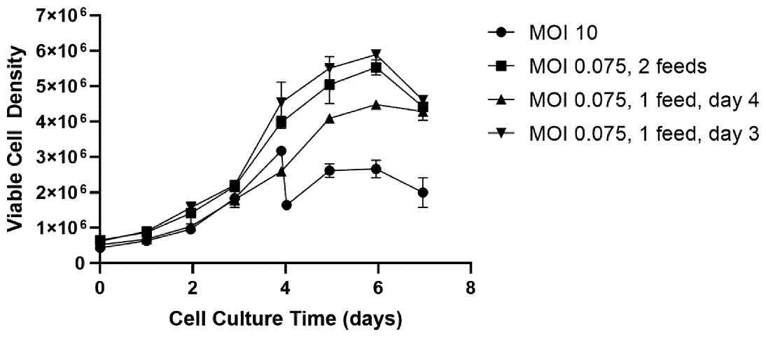

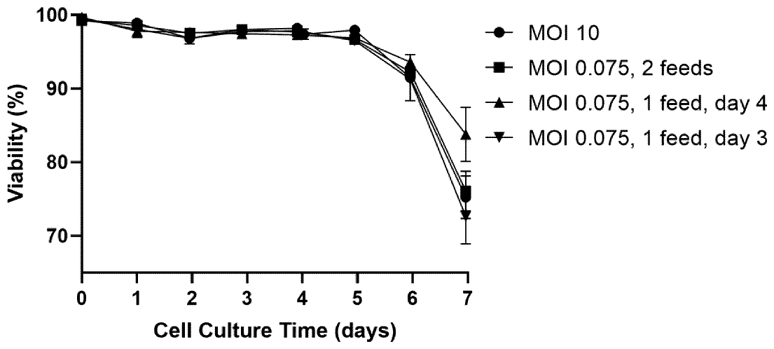

T-REx™ cells were seeded in 3 L bioreactors at 0.5×10⁶ viable cells per mL and subjected to high or low MOI adenovirus infection regimes based on those shown in FIG. 1 or FIG. 2. Briefly, for the high MOI infection regime, T-REx™ cells were grown until they reached a confluency of approximately 3-5×10⁶ cells/mL at which point they were diluted 1:1 and infected with adenovirus at an MOI of 10. The infected cells were fed with commercially available feed for HEK 293 cells when cell density reached 1×10⁶ viable cell per mL approximately 24 hours after infection. Separate cultures were harvested at 24, 48, and 72 hours after infection for assessment of viral titer.

In contrast, for the low MOI regime, T-REx™ cells were seeded in 3 L bioreactors at 0.7×10⁶ viable cells per mL and infected with adenovirus at an MOI of 0.075 approximately 24 hours after seeding. The infected cells were fed on day 4 and separate cultures were harvested at 72, 96, 120, 148, and 196 hours after seeding for assessment of viral titer.

During the process, viable cell density was determined using Vi-Cell and cell viability was measured using Vi-Cell.

After harvesting, qPCR was performed to determine viral genome titer with results giving gene copy per mL of sample. Methods of performing qPCR to determine viral genome titer are well known in the art. Briefly, virus DNA is extracted from virus particles and the copy number of virus DNA is determined by a PCR method using primers specific to virus transgene. In some cases, a plaque assay was used to determine the infectious titer with results showing infectious units per mL of sample. Methods of performing plaque assays to determine infectious titer are well known in the art. In some cases, the virus particle titer was measured using HPLC with results showing viral particles per mL of sample. Methods of using HPLC to quantitate adenovirus are well known in the art, for example, as described in Blanche et al. 2000; An improved anion-exchange HPLC method for the detection and purification of adenovirus particles; Gene Therapy 7, 1055-1062.

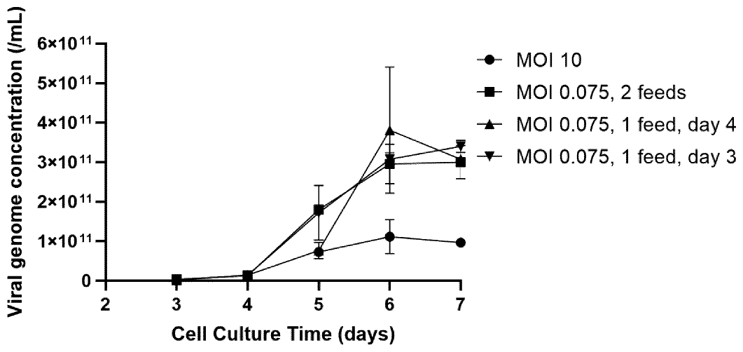

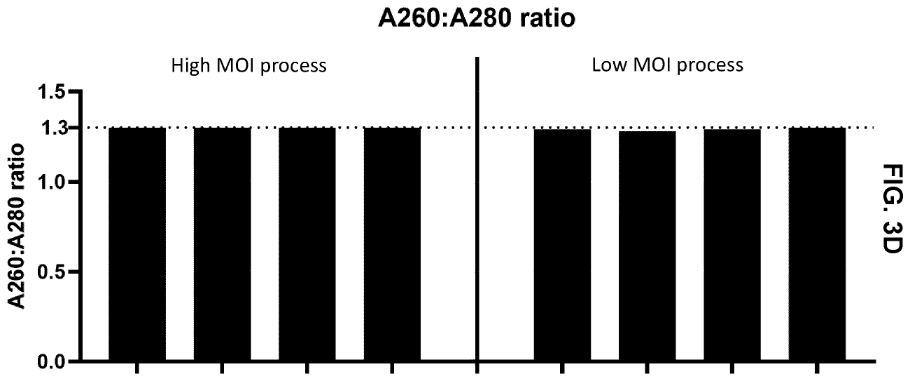

As shown in FIG. 3A, for cells infected at high MOI, viable cell density increased up until the day of infection at which point a large decrease in peak cell density was observed. Viable cell density recovered to about 2-3×10⁶ cells/mL approximately 24-48 hours after infection, before dropping to 1-2×10⁶ cells/mL at approximately 72 hours after infection.

Surprisingly, for cultures infected at low MOI, viable cell density increased until days 5-6 after seeding, reaching a peak cell density of approximately 4-6×10⁶ cells/mL. A slight decrease in peak cell density was observed on day 7 for these cells. Interestingly, cells fed on day 3 or on days 2 and 4 showed a higher peak cell density than those fed only on day 4, suggesting that the time at which cells are fed may impact peak cell density.

As shown in FIG. 3B, cell viability was not affected by any of the treatment regimes and stayed above 90% for all regimes tested until day 7 of culture. Surprisingly, FIG. 3C shows that cells infected at low MOI had higher viral titers than cells infected at high MOI. In particular, FIG. 3C shows that cells infected at a low MOI had a peak viral titer of approximately 3×10¹¹ GC/mL on day 6 of culture (5 days after infection), whereas those infected at a high MOI had a peak viral titer of approximately 1-1.5×10¹¹ GC/mL on day 6 of culture (2 days after infection). Importantly, product derived from the low MOI process had a comparable quality to that derived from the high MOI process, as shown in FIG. 3D.

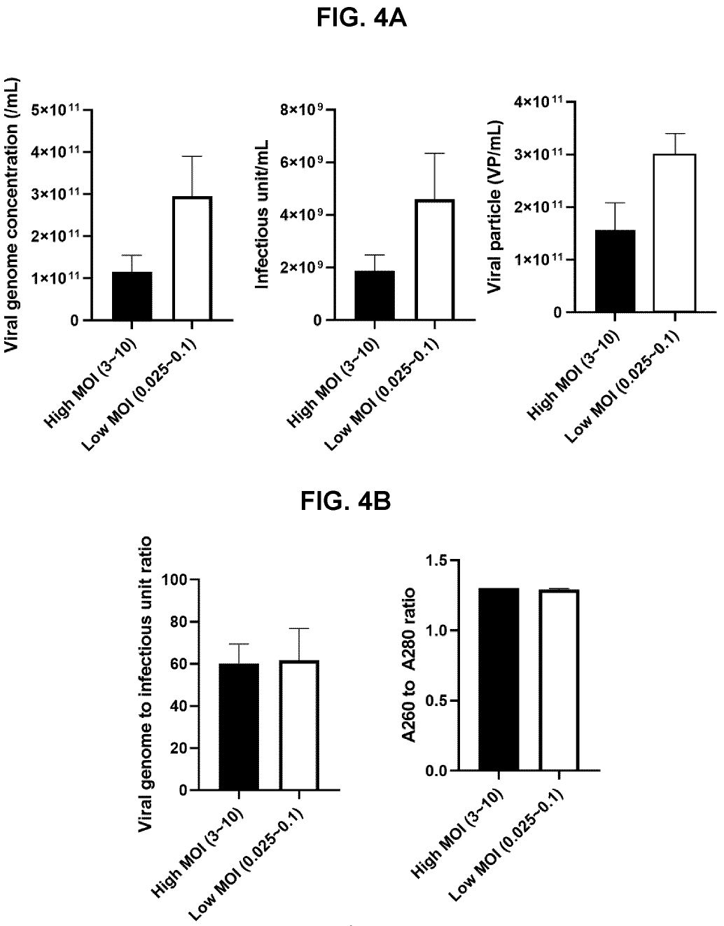

In summary, and as shown in FIG. 4A, use of a low MOI process results in a higher viral titer as marked by increased viral genome concentration, increased infectious units/mL and increased viral particle titer compared with use of a high MOI process. As shown in FIG. 4B, use of the low MOI process resulted in a similar viral genome to infectious unit ratio and similar product quality as the high MOI process.

Taken together, these results show that cells infected at low MOI reach a higher peak density and produce a higher viral titer than those infected at high MOI. As will be readily appreciated, use of low MOI infection considerably reduces the viral seed requirement compared with high MOI infections. Accordingly, due to the lower starting material required, infection of cells at low MOI represents a much more scalable method for the production of adenovirus than infection of cells at high MOI.

### Example 2—Effects of Infection at Low MOIs on Viral Titer

Next, the inventors tested the effect of a range of low MOIs on peak viable cell density, cell viability and virus titer. Briefly, T-REx™ cells were seeded in 3 L bioreactors at 0.7×10⁶ viable cells per mL and infected with adenovirus at an MOI of 0.026-0.270 approximately 24 hours after seeding. The infected cells were fed on day 2 and day 4 and separate cultures were harvested approximately 5, 6, and 7 days after seeding. As described previously, viable cell density and viability was measured for each of the cultures daily.

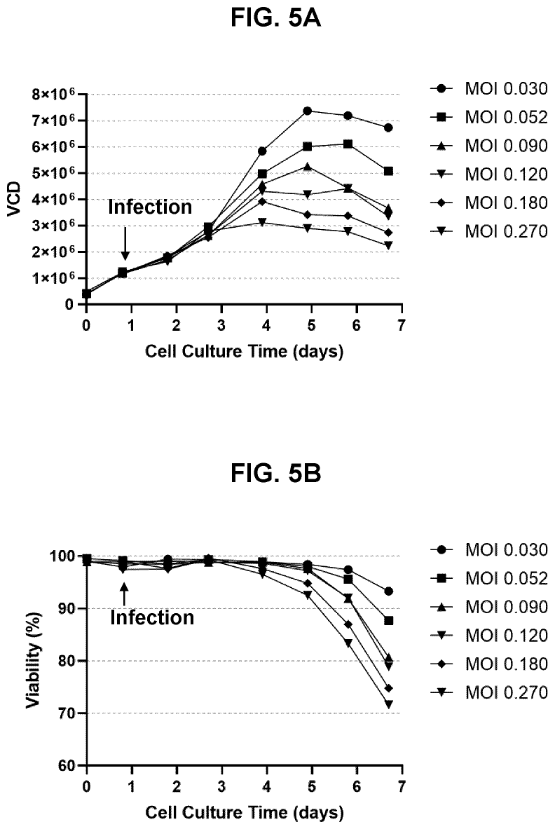

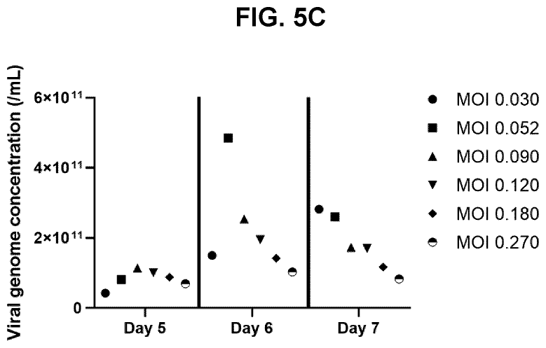

As shown in FIG. 5A, cells infected at a lower MOI had a higher peak viable cell density. Specifically, cells infected at an MOI of 0.026-0.030 had a peak cell density of about 7-8×10⁶ cells/mL, whereas cells infected at an MOI of 0.232-0.270 had a peak cell density of about 3×10⁶ cell/mL.

As shown in FIG. 5B, cell viability tended to decrease with increasing MOI. Thus, cells infected at an MOI of 0.026-0.030 had 95% viability even on day 7 of culture, whereas those infected at an MOI of 0.232-0.270 showed a significant drop off in viability beginning at approximately 4 days after infection, reaching approximately 75% viability on day 7 of culture.

Surprisingly, as shown in FIG. 5C, viral titer on day 7 (6 days after infection) was indirectly proportional to MOI. Accordingly, viral titer at day 7 was highest in cells infected at the lowest MOI tested and lowest in cells infected at the highest MOI tested. A similar pattern was observed for viral titer on day 6 (5 days after infection), with higher viral titers tending to be observed in cells infected with lower MOIs. In contrast, viral titer on day 5 (4 days after infection) was approximately equal at all MOIs tested apart from the lowest MOI which clearly showed the lowest viral titer at day 5. Of note, the highest viral titers were observed on day 6, and the highest viral titer on day 5 was lower than the lowest viral titer observed on either day 6 or day 7.

### Example 3—Effects of Cell Seeding Density on Viral Titer

The inventors next assessed viral titer at different initial cell seeding densities with infection on either day 0 or day 1. In brief, T-REx™ cells were seeded in amber 250 vessels at 0.5-1.2×10⁶ cells/mL and infected with adenovirus at target MOIs of 0.025 or 0.075 on day 0 or day 1 after seeding. Cells were cultured for up to 7 days post infection and cell culture was harvested for assessment of viral titer.

As shown in FIG. 6A, increasing cell density surprisingly increases viral titer for cultures infected at day 0 after cell seeding. Specifically, a cell seeding density of 0.5×10⁶ cells/mL resulted in a viral titer of <1×10¹¹ VG/mL when cultures were infected at an MOI of 0.025, whereas a cell seeding density of 1.2×10⁶ cells/mL resulted in a dramatically higher viral titer of approximately 4.5×10¹¹ VG/mL when cultures were infected at the same MOI. A similar effect was observed for cultures infected with an MOI of 0.075. FIG. 6B shows similar results for cultures infected at day 1 after cell seeding.

### Example 4—Effects of Cell Dilution on Viral Titer

The inventors next assessed whether cell dilution at the time of infection affects viral titer. In brief, T-REx™ cells were seeded in amber 250 vessels at 0.8×10⁶ cells/mL and infected with adenovirus at target MOIs of 0.025 or 0.075 on day 0 or day 1 after seeding. Some cultures were diluted at the time of infection. Cells were cultured for up to 5 days post infection and cell culture was harvested for assessment of viral titer.

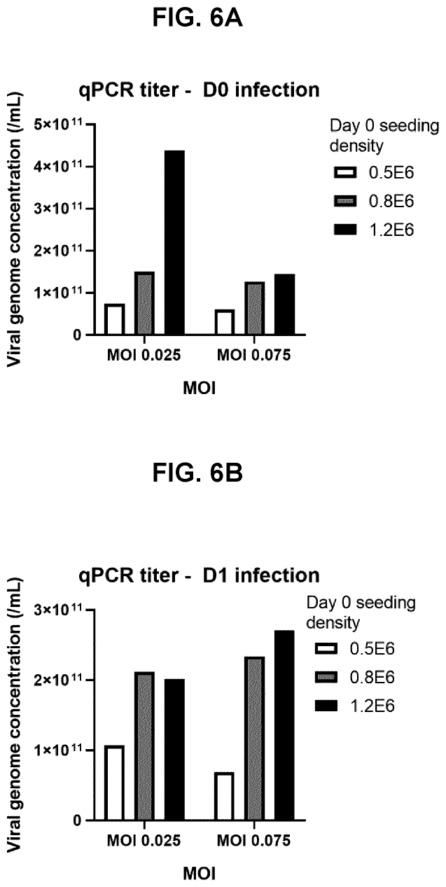

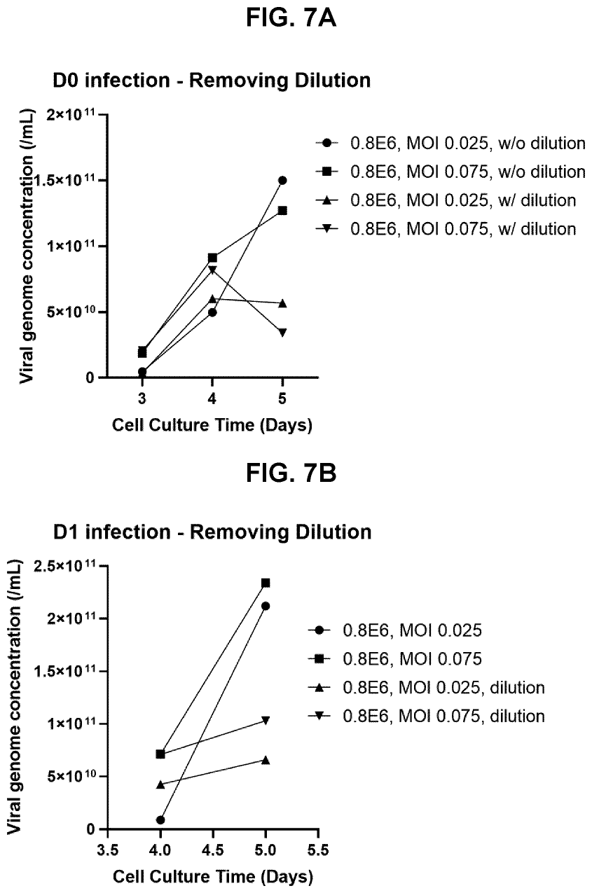

As shown in FIGS. 7A and 7B, diluting the cells at the time of infection drastically reduces viral titer. Specifically, FIG. 7A shows that viral titer is decreased from approximately 1.5×10¹¹ VG/mL to 5×10¹⁰ VG/mL if the cells are diluted at the time of infection on day 0 after cell seeding. Similarly, FIG. 7B shows that viral titer is decreased from approximately 2-2.5×10¹¹ VG/mL to 5×10¹⁰-1×10¹¹ VG/mL if the cells are diluted at the time of infection on day 1 after cell seeding.

### Example 5—Effects of Cell Culture Additives on Viral Titer

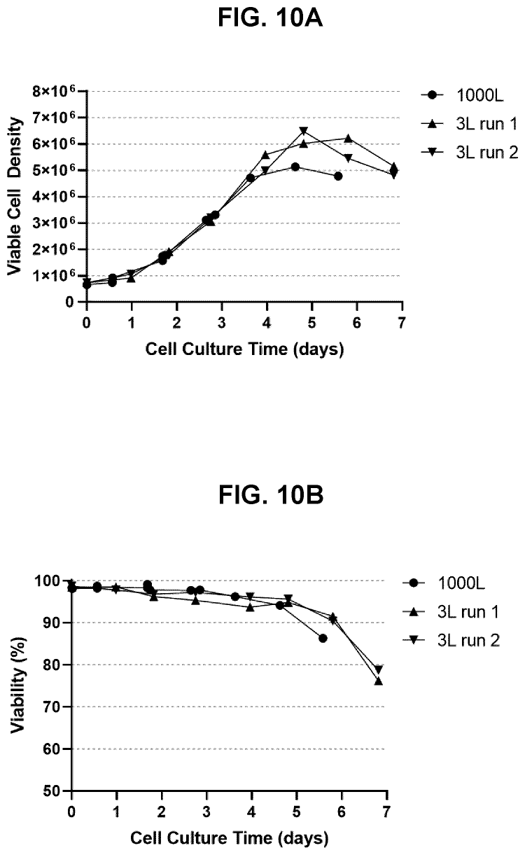

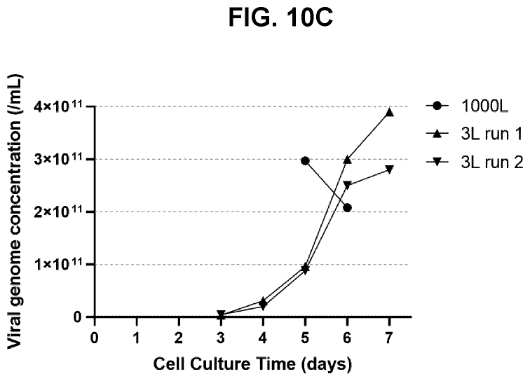

The inventors next assessed whether the cell culture additives DMSO, sodium butyrate or CaCl₂) could alter viral titer. In brief, T-REx™ cells were seeded in amber 250 vessels at 0.7×10⁶ viable cell per mL and infected with adenovirus at target MOIs of 0.075 on day 1 after seeding. Cell culture additives were added as shown in FIG. 10 on day 4 or day 5 after seeding. Cells were cultured for up 6 days post infection and cell culture was harvested for assessment of viral titer.

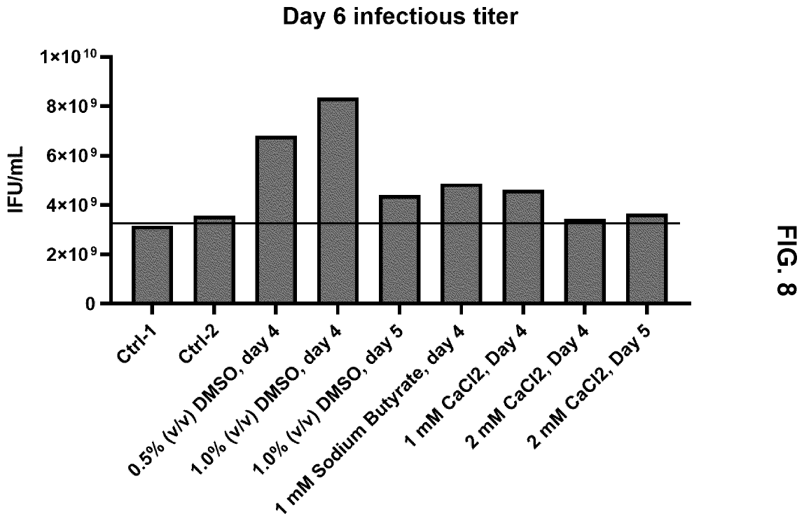

As shown in FIG. 8, addition of 0.5% DMSO at day 4 or 1% DMSO at day 4 or day 5 increased the viral titer compared with control. Similarly, addition of 1 mM sodium butyrate at day 4 also increased viral titer compared with control. Finally, addition of 1 mM CaCl₂) on day 4, but not 2 mM CaCl₂) on day 4 or day 5 increased viral titer compared to control.

### Example 6—Effect of Temperature Shift on Viral Titer

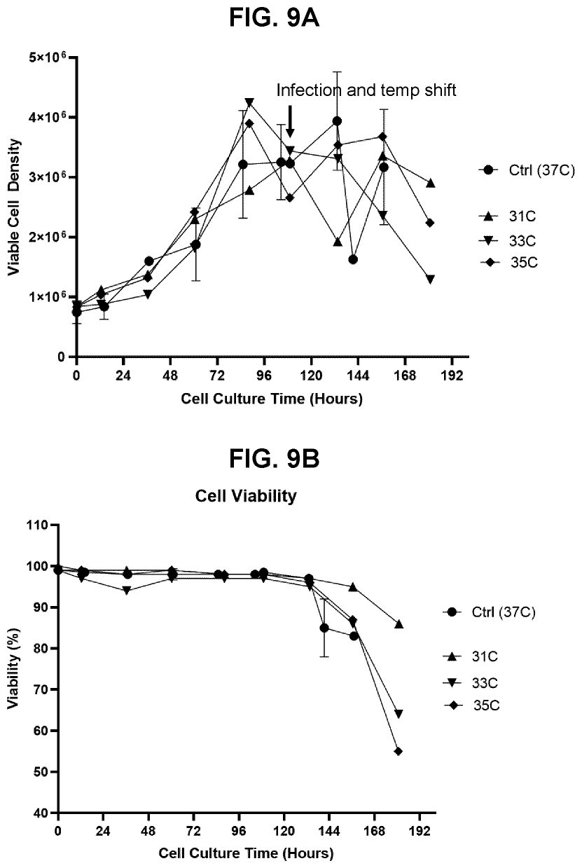

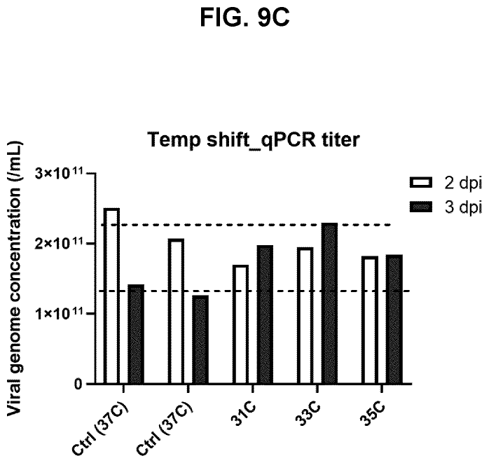

The inventors next assessed whether a temperature shift during the infection process could alter peak cell density, cell viability and viral titer. Accordingly, cells were cultured at 37° C. until infection, and temperature was shifted to 31° C., 33° C., or 35° C. approximately 3 hours after addition of adenovirus to the culture. As shown in FIG. 9A, cell density of all samples closely mimicked at least one of the control cultures which were cultured at 37° C. throughout the process.

Surprisingly, FIG. 9B shows that cell viability was higher in cultures which were subjected to a temperature shift, with a bigger temperature shift associated with increased cell viability. In particular, cultures that were shifted to 31° C. showed a cell viability of approximately 90% even after 192 hours of culture, whereas cultures that were shifted to 33° C. showed a cell viability of approximately 65% and cultures that were shifted to 35° C. showed a cell viability of approximately 55%, similar to that observed in cultures that were not subjected to a temperature shift.

Finally, FIG. 9C shows that viral titer 2 days post infection was highest in cultures that were not subjected to a temperature shift. However, viral titer in these cultures dramatically decreased between 2-3 days post infection. In one control culture, viral titer decreased from approximately 2.5×10¹¹ VG/mL at 2 days post infection to <1.5×10¹¹ VG/mL at 3 days post infection. Surprisingly, viral titer in cultures subjected to a temperature shift increased between 2-3 days post infection. In particular, viral titer in cultures subjected to a 33° C. temperature shift increased to >2×10¹¹ VG/mL at 3 days post infection.

### Example 7—Scalability

To show that the low MOI process is scalable, the inventors next compared the process in 1000 L and 3 L bioreactors. As shown in FIGS. 10A and 10B, peak cell density and cell viability in 1000 L bioreactors reaches an acceptable level approximating that seen in 3 L bioreactors up until day 5 of culture. Surprisingly, as shown in FIG. 10C, viral titer was approximately 3× higher in cultures in 1000 L bioreactors at day 5 compared with those in 3 L bioreactors.

Taken together, these results show that the use of low MOI infection provides a highly scalable and efficient process for the production of adenovirus.

1. A method of producing an adenovirus for use in a vaccine, the method comprising:
(a) adding an adenovirus to a cell population in culture at an MOI insufficient for infection of all the cells in the cell population;
(b) culturing the cell population under conditions which are permissive for infection of the cell population with the adenovirus to provide a cell population comprising adenovirus-infected cells;
(c) culturing the cell population comprising adenovirus-infected cells under conditions which are permissive for replication of the adenovirus; and
(d) harvesting the adenovirus from the culture.

2. The method of claim 1, wherein step (a) comprises adding the adenovirus to the cell population at an MOI of about 0.01-1, preferably at an MOI of about 0.025-0.4, most preferably at an MOI of about 0.1.

3. The method of claim 1 or claim 2, wherein step (a) comprises adding the adenovirus to the cell population about 0-48 hours after inoculation of a cell culture medium with the cell population.

4. The method of claim 3, wherein step (a) comprises adding the adenovirus to the cell population about 24 hours after inoculation of the cell culture medium with the cell population.

5. The method of any one of the preceding claims, wherein the method is characterised by a first infection and a second infection, wherein the first infection provides a first fraction of adenovirus-infected cells and is induced by adding the adenovirus to the cell population, and wherein the second infection provides a second fraction of adenovirus-infected cells and is induced by adenovirus released into the culture by the first fraction of adenovirus-infected cells.

6. The method of any one of the preceding claims, wherein the method comprises switching the temperature to which the cell population is exposed from a first temperature to a second temperature, wherein the first and second temperatures are permissive for infection of the cell population with the adenovirus.

7. The method of claim 6, wherein exposing the cell population to the second temperature increases the stability of the adenovirus in the culture.

8. The method of claim 6 or claim 7, wherein exposing the cell population to the second temperature decreases the adenovirus particle:infectious particle ratio in the culture.

9. The method of any one of claims 6-8, wherein the second temperature is permissive for growth of the cell population.

10. The method of any one of claims 6-9, wherein the conditions which are permissive for infection of the cell population with the adenovirus comprise culturing the cell population at the first temperature and the conditions which are permissive for replication of the adenovirus comprise culturing the cell population at the second temperature.

11. The method of any one of claims 6-10, wherein the method comprises switching the temperature to which the cell population is exposed from the first temperature to the second temperature about 48-96 hours after adding the adenovirus to the cell population.

12. The method of claim 11, wherein the method comprises switching the temperature to which the cell population is exposed from the first temperature to the second temperature about 72 hours after adding the adenovirus to the cell population.

13. The method of any one of claims 6-12, wherein the first temperature is higher than the second temperature.

14. The method of any one of claims 6-13, wherein the first temperature is about 31-40° C., preferably about 35-38° C., most preferably about 37° C.

15. The method of any one of claims 6-14, wherein the second temperature is about 27-40, preferably about 31-35° C., most preferably about 33° C.

16. The method of any one of claims 6-15, wherein the cell population is cultured at the first temperature for at least about 72 hours.

17. The method of claim 16, wherein the cell population is cultured at the first temperature for at least about 96 hours.

18. The method of any one of claims 6-17, wherein the cell population is cultured at the second temperature for at least about 48 hours.

19. The method of any one of claims 6-18, wherein the cell population is cultured at the second temperature until the adenovirus is harvested from the culture.

20. The method of any one of the preceding claims, wherein the cell population is cultured in a bioreactor having a capacity of at least about 1 L, preferably at least about 1.2 L, at least about 3 L, about 50 L, about 1000 L, about 2000 L, about 3000 L, or about 5000 L, most preferably at least about 2000 L.

21. The method of any one of the preceding claims, wherein the method does not comprise a step of replacing or adding cell culture medium to the cell population after adding the adenovirus to the cell population.

22. The method of any one of the preceding claims, wherein the method comprises adding a cell culture additive to the cell population.

23. The method of claim 22, wherein the cell culture additive comprises DMSO, sodium butyrate and/or CaCl₂.

24. The method of claim 22 or 23, wherein after adding the cell culture additive to the cell population, the cell population is exposed to DMSO, preferably about 0.5% or 1% DMSO.

25. The method of any one of claims 22-24, wherein after adding the cell culture additive to the cell population, the cell population is exposed to sodium butyrate, preferably about 1 mM sodium butyrate.

26. The method of any one of claims 22-25, wherein after adding the cell culture additive to the cell population, the cell population is exposed to CaCl₂, preferably about 2 mM CaCl₂.

27. The method of any one of claims 22-26, wherein the method comprises adding the cell culture additive to the cell population during step (c).

28. The method of any one of claims 22-27, wherein the method comprises adding the cell culture additive to the cell population about 72-120 hours after adding the adenovirus to the cell population.

29. The method of any one of the preceding claims, wherein the method comprises adding a feed to the cell population.

30. The method of claim 29, wherein the method comprises adding the feed to the cell population about 24-48 hours after adding the adenovirus to the cell population.

31. The method of any one of claims 29-30, wherein the method comprises adding the feed to the cell population at least every 48 hours.

32. The method of claim 30 or claim 31, wherein the method comprises adding the feed to the cell population at a final concentration of about 5% v/v.

33. The method of any one of the preceding claims, wherein the cell population is complementary to the adenovirus added to the cell population.

34. The method of any one of the preceding claims, wherein the cell population comprises mammalian cells.

35. The method of claim 34, wherein the mammalian cells express an adenovirus replication factor.

36. The method of claim 34 or claim 35, wherein the cell population comprises HEK cells.

37. The method of any one of claims 34-36, wherein the cell population comprises T-REx cells.

38. The method of claim 34, wherein the cell population consists of T-REx cells.

39. The method of any one of the preceding claims, wherein the adenovirus is a replication deficient adenovirus.

40. The method of any one of the preceding claims, wherein the adenovirus is a simian adenovirus.

41. The method of claim 40, wherein the simian adenovirus is a replication-deficient simian adenovirus.

42. The method of claim 41, wherein the replication-deficient simian adenovirus is ChAdOx1, ChAdOx2, ChAdOx3, or ChAd63, preferably wherein the replication-deficient simian adenovirus is ChAdOx1.

43. The method of any one of the preceding claims, wherein the adenovirus is not a human adenovirus.

44. The method of any one of the preceding claims, wherein the adenovirus encodes nCov-19 spike protein.

45. The method of any one of the preceding claims, wherein the step of harvesting adenovirus from the culture is performed about 96-144 hours after adding the adenovirus to the cell population.

46. The method of claim 45, wherein the step of harvesting adenovirus from the culture is performed about 120 hours after adding the adenovirus to the cell population.

47. The method of any one of the preceding claims, wherein the step of harvesting the adenovirus from the culture comprises lysing the cells of the cell population and harvesting adenovirus from the cell lysate of the cell population.

48. The method of any one of the preceding claims, wherein the step of harvesting the adenovirus from the culture comprises harvesting adenovirus from the cell culture medium in which the cell population was cultured.

49. The method of any one of the preceding claims, wherein prior to step (a), the method comprises seeding cells in a cell culture vessel.

50. The method of claim 49, wherein prior to step (a), the method comprises seeding cells in a cell culture vessel at an initial cell density of at least about 0.5×10⁶ cells/mL, preferably at least about 0.8×10⁶ cells/mL, most preferably at least about 1.2×10⁶ cells/mL to provide the cell population in culture.

51. The method of any one of the preceding claims, wherein the method comprises adding the adenovirus to a cell population in culture having a viable cell density of at least about 1×10⁶ cells/mL.

52. The method of any one of the preceding claims, wherein the peak virus titer is about 6-8 days after addition of the adenovirus to the cell population.

53. The method of any one of the preceding claims, wherein the conditions which are permissive for infection of the cell population with the adenovirus are conditions permissive for cell growth.

54. The method of any one of the preceding claims, wherein the conditions which are permissive for replication of the adenovirus are conditions permissive for cell growth.

55. The method of any one of the preceding claims, wherein the vaccine is a COVID-19 vaccine.

56. A method of producing an adenovirus for use in a vaccine, the method comprising culturing a cell population comprising a first fraction of adenovirus-infected cells under conditions which are permissive for infection of a second fraction of the cell population with the adenovirus, wherein the second fraction of the cell population is infected by adenovirus released into the culture by the first fraction of adenovirus-infected cells.

57. The method of claim 56, wherein prior to culturing the cell population comprising a first fraction of adenovirus-infected cells under conditions which are permissive for infection of a second fraction of the cell population with the adenovirus, the method comprises culturing the cell population under conditions which are permissive for infection of the first fraction of cells in the cell population with the adenovirus.

58. The method of claim 57, wherein prior to culturing the cell population under conditions which are permissive for infection of the first fraction of cells in the cell population with the adenovirus, the method comprises adding the adenovirus to the cell population in culture.

59. The method of any one of claims 56-58, further comprising culturing the cell population comprising adenovirus-infected cells under conditions which are permissive for replication of the adenovirus.

60. The method of any one of claims 56-59, further comprising harvesting the adenovirus from the culture.

61. The method of any one of claims 56-60, wherein the method comprises:
(a) adding an adenovirus to a cell population in culture;
(b) culturing the cell population under conditions which are permissive for infection of a first fraction of cells in the cell population with the adenovirus;
(c) culturing the cell population comprising the first fraction of infected cells under conditions which are permissive for infection of a second fraction of cells in the cell population with the adenovirus, wherein the second fraction of cells is infected by adenovirus released into the culture by the first fraction of infected cells;
(d) culturing the cell population comprising adenovirus-infected cells under conditions which are permissive for replication of the adenovirus; and
(d) harvesting the adenovirus from the culture.

62. The method of any one of claims 56-61, wherein the conditions which are permissive for infection of the first fraction of the cell population are conditions permissive for cell growth.

63. The method of any one of claims 56-62, wherein the conditions which are permissive for infection of the second fraction of the cell population are conditions permissive for cell growth.

64. The method of any one of claims 56-63, wherein the conditions which are permissive for infection of the first and second fraction of the cell population are the same.

65. The method of any one of claims 56-64, wherein the conditions which are permissive for infection of the first and second fraction of the cell population are different.

66. The method of any one of claims 56-65, further comprising culturing the cell population comprising the first and second fraction of infected cells under conditions which are permissive for infection of a third fraction of cells in the cell population with the adenovirus, wherein the third fraction of cells is infected by adenovirus released by the first and/or second fraction of infected cells.

67. A method of producing an adenovirus for use in a vaccine, the method comprising:
(a) adding an adenovirus to a cell population in culture;
(b) culturing the cell population under conditions which are permissive for infection of the cell population to provide a cell population comprising adenovirus-infected cells;
(c) culturing the cell population comprising adenovirus-infected cells under conditions which are permissive for replication of the adenovirus; and
(d) harvesting adenovirus from the culture about 96-144 hours after adding adenovirus to the cell population.

68. A method of producing an adenovirus for use in a vaccine, the method comprising:
(a) seeding cells in a cell culture vessel at an initial cell density of at least 0.5×10⁶ cells/mL to provide a cell population in culture;
(b) adding an adenovirus to the cell population in culture;
(c) culturing the cell population under conditions which are permissive for infection of the cell population to provide a cell population comprising adenovirus-infected cells;
(d) culturing the cell population comprising adenovirus-infected cells under conditions which are permissive for replication of the adenovirus; and
(e) harvesting adenovirus from the culture.

69. A method of producing an adenovirus for use in a vaccine, the method comprising:
(a) adding an adenovirus to a cell population in culture having a viable cell density of at least about 1×10⁶ cells/mL;
(b) culturing the cell population under conditions which are permissive for infection of the cell population with the adenovirus to provide a cell population comprising adenovirus-infected cells;
(c) culturing the cell population comprising adenovirus-infected cells under conditions which are permissive for replication of the adenovirus; and
(d) harvesting the adenovirus from the culture.

70. A method of producing of an adenovirus for use in a vaccine, the method comprising:
(a) adding an adenovirus to a cell population in culture having a viable cell density of at least about 1×10⁶ cells/mL at an MOI insufficient for infection of all the cells in the cell population;
(b) culturing the cell population under conditions which are permissive for infection of the cell population to provide a cell population comprising adenovirus-infected cells;
(c) culturing the cell population comprising adenovirus-infected cells under conditions which are permissive for replication of the adenovirus; and
(d) harvesting adenovirus from the culture about 96-144 after adding adenovirus to the cell population,
wherein the method comprises switching the temperature to which the cell population is exposed from a first temperature to a second temperature, wherein the first and second temperatures are permissive for infection of the cell population with the adenovirus.

71. A method of producing an adenovirus for use in a vaccine, the method comprising:
(a) adding an adenovirus to a cell population in culture having a viable cell density of at least about 1×10⁶ cells/mL at an MOI insufficient for infection of all the cells in the cell population;
(b) culturing the cell population under conditions which are permissive for infection of a first fraction of cells in the cell population with the adenovirus;
(c) culturing the cell population comprising the first fraction of infected cells under conditions which are permissive for infection of a second fraction of cells in the cell population with the adenovirus, wherein the second fraction of cells is infected by adenovirus released into the culture by the first fraction of infected cells;
(d) culturing the cell population comprising adenovirus-infected cells under conditions which are permissive for replication of the adenovirus; and
(e) harvesting the adenovirus from the culture about 96-144 hours after adding adenovirus to the cell population,
wherein the method comprises switching the temperature to which the cell population is exposed from a first temperature to a second temperature, wherein the first and second temperatures are permissive for infection of the cell population with the adenovirus.

72. A method for preparing a vaccine comprising an adenovirus, the method comprising:
(a) adding an adenovirus to a cell population in culture at an MOI insufficient for infection of all the cells in the cell population;
(b) culturing the cell population under conditions which are permissive for infection of the cell population to provide a cell population comprising adenovirus-infected cells;
(c) culturing the cell population comprising adenovirus-infected cells under conditions which are permissive for replication of the adenovirus;
(d) harvesting the adenovirus from the culture;
(e) purifying the adenovirus; and
(f) preparing a vaccine comprising the purified adenovirus.

73. A method for preparing a vaccine comprising an adenovirus, the method comprising:
(a) culturing a cell population comprising a first fraction of adenovirus-infected cells under conditions which are permissive for infection of a second fraction of the cell population with the adenovirus, wherein the second fraction of the cell population is infected by adenovirus released into the culture by the first fraction of infected cells;
(b) harvesting the adenovirus from the culture;
(c) purifying the adenovirus; and
(d) preparing a vaccine comprising the purified adenovirus.

74. A method for preparing a vaccine comprising an adenovirus, the method comprising:
(a) adding an adenovirus to a cell population in culture;
(b) culturing the cell population under conditions which are permissive for infection of the cell population to provide a cell population comprising adenovirus-infected cells;
(c) culturing the cell population comprising adenovirus-infected cells under conditions which are permissive for replication of the adenovirus;
(d) harvesting adenovirus from the culture about 96-144 hours after adding adenovirus to the cell population;
(e) purifying the adenovirus; and
(f) preparing a vaccine comprising the purified adenovirus.

75. A method for preparing a vaccine comprising an adenovirus, the method comprising:
(a) seeding cells in a cell culture vessel at an initial cell density of at least 0.5×10⁶ cells/mL to provide a cell population in culture;
(b) adding an adenovirus to the cell population in culture;
(c) culturing the cell population under conditions which are permissive for infection of the cell population to provide a cell population comprising adenovirus-infected cells;
(d) culturing the cell population comprising adenovirus-infected cells under conditions which are permissive for replication of the adenovirus;
(e) harvesting adenovirus from the culture;
(f) purifying the adenovirus; and
(g) preparing a vaccine comprising the purified adenovirus.

76. A method for preparing a vaccine comprising an adenovirus, the method comprising:
(a) adding an adenovirus to a cell population in culture having a viable cell density of at least about 1×10⁶ cells/mL;
(b) culturing the cell population under conditions which are permissive for infection of the cell population with the adenovirus to provide a cell population comprising adenovirus-infected cells;
(c) culturing the cell population comprising adenovirus-infected cells under conditions which are permissive for replication of the adenovirus;
(d) harvesting the adenovirus from the culture;
(e) purifying the adenovirus; and
(f) preparing a vaccine comprising the purified adenovirus.

77. A method for preparing a vaccine comprising an adenovirus, the method comprising:
(a) adding an adenovirus to a cell population in culture having a viable cell density of at least about 1×10⁶ cells/mL at an MOI insufficient for infection of all the cells in the cell population;
(b) culturing the cell population under conditions which are permissive for infection of the cell population to provide a cell population comprising adenovirus-infected cells;
(c) culturing the cell population comprising adenovirus-infected cells under conditions which are permissive for replication of the adenovirus;
(d) harvesting adenovirus from the culture about 96-144 hours after adding adenovirus to the cell population;
(e) purifying the adenovirus; and
(f) preparing a vaccine comprising the purified adenovirus,
wherein the method comprises switching the temperature to which the cell population is exposed from a first temperature to a second temperature, wherein the first and second temperatures are permissive for infection of the cell population with the adenovirus.

78. A method for preparing a vaccine comprising an adenovirus, the method comprising:
(a) adding an adenovirus to a cell population in culture having a viable cell density of at least about 1×10⁶ cells/mL at an MOI insufficient for infection of all the cells in the cell population;
(b) culturing the cell population under conditions which are permissive for infection of a first fraction of cells in the cell population with the adenovirus;
(c) culturing the cell population comprising the first fraction of infected cells under conditions which are permissive for infection of a second fraction of cells in the cell population with the adenovirus, wherein the second fraction of cells is infected by adenovirus released into the culture by the first fraction of infected cells;
(d) culturing the cell population comprising adenovirus-infected cells under conditions which are permissive for replication of the adenovirus; and
(e) harvesting the adenovirus from the culture about 96-144 hours after adding adenovirus to the cell population,
(f) purifying the adenovirus; and
(g) preparing a vaccine comprising the purified adenovirus,
wherein the method comprises switching the temperature to which the cell population is exposed from a first temperature to a second temperature, wherein the first and second temperatures are permissive for infection of the cell population with the adenovirus.

79. A method for increasing the yield of an adenovirus during production of the adenovirus, the method comprising culturing a cell population in culture in the presence of an adenovirus at a first temperature and switching the temperature to which the cell population is exposed to a second temperature, wherein the first and second temperatures are permissive for infection of the cell population with the adenovirus.

80. An adenovirus for use in a vaccine, obtainable by the method of any one of claims 1-71.

81. A vaccine comprising an adenovirus, obtainable by the method of any one of claims 72-79.


SEQUENCE LISTING (2 sequences)

SEQ ID NO: 1 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1304 aa, Severe acute respiratory syndrome-related coronavirus
MDAMKRGLCCVLLLCGAVFVSASQEIHARFRRFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT